(19) United States
(12) Patent Application Publication
(10) Pub. No.: US 2026/0226540 A1
(43) Pub. Date: Aug. 6, 2026

(54) SEQUENCING REAGENTS

(71) Applicant: ULTIMA GENOMICS, INC., Fremont, CA (US)

(72) Inventors: Charles FRANCAVILLA, Fremont, CA (US); Daniel MAZUR, San Diego, CA (US); Abhisek RAY, Santa Clara, CA (US); Maodie WANG, Fremont, CA (US); Dancan NJERI, Newark, CA (US); Theo NIKIFOROV, Carlsbad, CA (US); Emma ZANARDI, Carlsbad, CA (US); Kevin HEINEMANN, Carlsbad, CA (US)

(21) Appl. No.: 19/458,032

(22) Filed: Jan. 23, 2026

Related U.S. Application Data
(63) Continuation of application No. PCT/US2024/040440, filed on Jul. 31, 2024.
(60) Provisional application No. 63/530,231, filed on Aug. 1, 2023.
(60) Provisional application No. 63/539,321, filed on Sep. 19, 2023.

(51) Int. Cl. C12Q 1/6869

(52) U.S. Cl. CPC C12Q 1/6869

(57) ABSTRACT

Provided herein are sequencing reagents. The sequencing reagent may comprise a labelling reagent or a labelled substrate comprising a polyethylene glycol (PEG) polymer. The sequencing reagent may comprise a labelling reagent or a labelled substrate comprising a protein.

### CROSS-REFERENCE

This application is a continuation of International Patent Application No. PCT/US2024/040440, filed Jul. 31, 2024, which claims the benefit of U.S. Provisional Patent Application Nos. 63/530,231, filed Aug. 1, 2023, and 63/539,321, filed Sep. 19, 2023, each of which is entirely incorporated by reference herein.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

Sequencing a template nucleic acid molecule may comprise probing the template nucleic acid molecule with a reagent and detecting a signal from the reagent and/or from the template nucleic acid molecule. For example, the reagent may be labeled, and a signal from the reagent may be detected. For example, the reagent may be a labeled nucleotide reagent.

### SUMMARY

Recognized herein is the need for systems, methods, processes, and compositions for increasing the efficiency, sensitivity, and accuracy of methods for analyzing and/or processing nucleic acid samples, for example template or sample nucleic acid molecules. Provided herein are systems, methods, and compositions comprising sequencing reagents that may be used during a sequencing reaction. The sequencing reaction may be a sequencing-by-synthesis reaction, such as a nucleotide incorporation reaction, or any probing reaction. The sequencing reagents may comprise a labelling reagent. The sequencing reagents may comprise a labelled substrate, such as a labelled nucleotide. The present disclosure may be advantageous to improve sequencing results.

Provided herein are nucleotide reagents that comprises a (i) a nucleotide, (ii) a linker, and (iii) a label, such as a dye, wherein the linker comprises polyethylene glycol (PEG) or modified PEG.

In an aspect, provided is a labeling reagent, comprising: a fluorescent dye moiety; and a linker that is connected to the fluorescent dye moiety and configured to couple to a substrate, wherein the linker comprises a polymer linker moiety selected from the group consisting of:

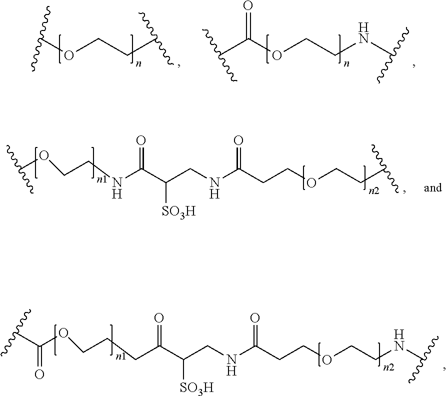

where each of n, n1, and n2 is a positive integer.

In another aspect, provided is a labeling reagent, comprising: a fluorescent dye moiety; and a linker that is connected to the fluorescent dye moiety and configured to couple to a substrate, wherein the linker comprises: (i) a cleavable moiety selected from the group consisting of:

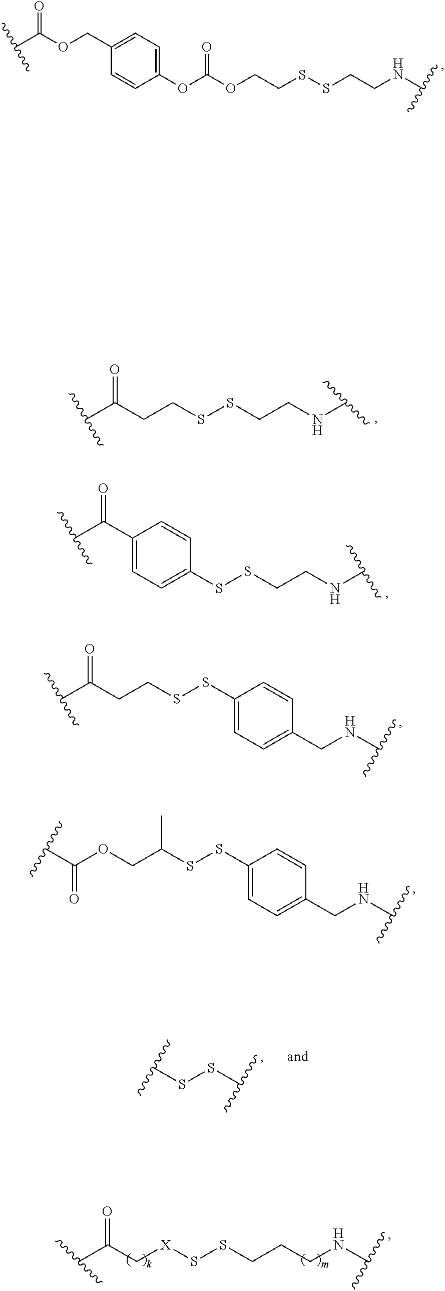

where X is a bond or 1,4-phenylene, where k is 0, 1, or 2, and where m is 0, 1, 2, 3, or 4; and (ii) a polymer linker moiety selected from the group consisting of:

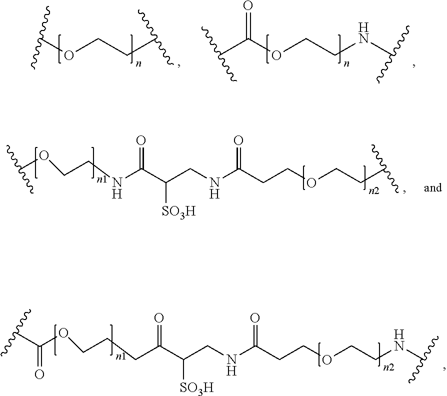

where each of n, n1, and n2 is a positive integer.

In some embodiments, n or (n1+n2) is 8 or greater. In some embodiments, n or (n1+n2) is 75 or greater. In some embodiments, n or (n1+n2) is 100 or greater.

In some embodiments, the linker further comprises a moiety selected from the group consisting of:

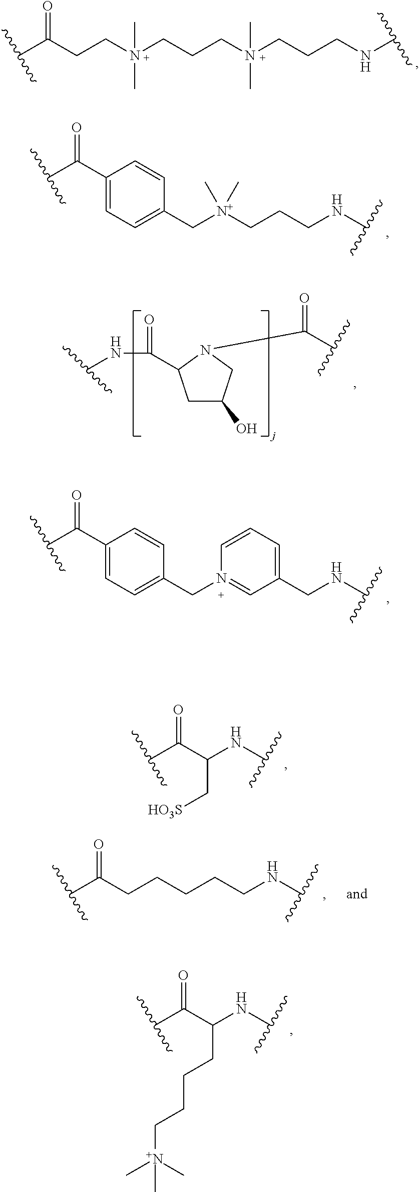

where j is a positive integer.

In some embodiments, the linker further comprises one or more glycine moieties.

In some embodiments, the linker provides an average physical separation between the fluorescent dye moiety and the substrate of at least 30 Angstroms (Å). In some embodiments, the linker provides an average physical separation between the fluorescent dye moiety and the substrate of at least 60 Angstroms (Å).

In some embodiments, the linker further comprises a cleavable group selected from the group consisting of an azidomethyl group, a disulfide bond, a hydrocarbyldithiomethyl group, and a 2-nitrobenzyloxy group.

In some embodiments, the cleavable group is cleavable by application of one or more members of the group consisting of tris(2-carboxyethyl) phohsphine (TCEP), dithiothreitol (DTT), tetrahydropyranyl (THP), ultraviolet (UV) light, and a combination thereof.

In some embodiments, the substrate is a nucleotide, polynucleotide, protein, lipid, cell, saccharide, polysaccharide, or antibody.

In some embodiments, the substrate is a nucleotide and the labeling reagent is configured to attach to a nucleobase of the nucleotide.

In some embodiments, the substrate is a protein.

In some embodiments, the linker comprises two or more linker branches each coupled to a dendrimer core, wherein each of the two or more linker branches comprises

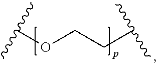

where p is a positive integer, and wherein the dendrimer core is attached to the fluorescent dye moiety.

In some embodiments, the a linker branch of the two or more linker branches is terminated by a water soluble group. In some embodiments, the water soluble group comprises sulfonic acid. In some embodiments, the water soluble group comprises three sulfonic acid moieties.

In some embodiments, the each of the two or more linker branches is terminated by a respective water soluble group.

In some embodiments, p is at least 8.

In another aspect, provided is a labeled substrate, comprising: a substrate; and a labeling reagent of any one of the above labeling reagent embodiments, wherein the labeling reagent is coupled to the substrate.

In some embodiments, the substrate is a nucleotide, polynucleotide, protein, lipid, cell, saccharide, polysaccharide, or antibody.

In some embodiments, the substrate is a nucleotide and the labeling reagent is configured to attach to a nucleobase of the nucleotide.

In some embodiments, the substrate is a protein.

In another aspect, provided is a labeling reagent, comprising: a protein; a predetermined substrate attachment site configured to attach the protein to a substrate; and a predetermined optical moiety attachment site configured to attach the protein to an optical moiety.

In some embodiments, the labeling reagent comprises a single predetermined substrate attachment site.

In some embodiments, the labeling reagent comprises at least two predetermined optical moiety attachment sites. In some embodiments, the labeling reagent comprises at least three predetermined optical moiety attachment sites.

In some embodiments, two of the at least two predetermined optical moiety attachment sites are separated by a polyproline or at least one EAAAK linker moiety.

In some embodiments, the predetermined substrate attachment site is an amino acid residue native to the protein.

In some embodiments, the predetermined substrate attachment site is an engineered amino acid residue that is mutated within or coupled to the protein.

In some embodiments, the predetermined substrate attachment site is selected from the group consisting of glycine, methionine, arginine, cysteine, tryptophan, tyrosine, lysine, serine and histidine amino acid residues.

In some embodiments, the predetermined substrate attachment site is selected from glycine, cysteine, and serine amino acid residues.

In some embodiments, the predetermined optical moiety attachment site is an amino acid residue native to the protein.

In some embodiments, the predetermined optical moiety attachment site is an engineered amino acid residue that is mutated within or coupled to the protein.

In some embodiments, the predetermined optical moiety attachment site is selected from the group consisting of glycine, methionine, arginine, cysteine, tryptophan, tyrosine, lysine, serine and histidine amino acid residues.

In some embodiments, the predetermined optical moiety attachment site is selected from cysteine and lysine amino acid residues.

In some embodiments, the protein is an engineered protein that mutates or removes at least one native amino acid residue.

In some embodiments, the protein comprises at most 500 amino acid residues.

In some embodiments, the protein has a molecular mass of at most 50 kilodaltons (kDa).

In some embodiments, a distance between the N-terminus and the C-terminus of the protein is at least about 25 Angstroms (Å).

In some embodiments, the substrate attachment site is disposed within at most 10 amino acid residues of the N-terminus of the protein.

In some embodiments, the optical moiety attachment site is disposed within at most 10 amino acid residues of the C-terminus of the protein.

In some embodiments, the protein is a small ubiquitin-like modifier (SUMO) proteins, maltose-binding proteins (MBP), or thioredoxin.

In another aspect, provided is a labeled substrate, comprising: a substrate; an optical moiety; and the labeling reagent of any one of the above labeling reagent embodiments, wherein the substrate is attached to the substrate attachment site and the optical moiety is attached to the optical moiety attachment site.

In some embodiments, the substrate comprises a nucleotide.

In some embodiments, the labeled substrate further comprises a cleavable group between the substrate and the substrate attachment site.

In some embodiments, the cleavable group is selected from the group consisting of an azidomethyl group, a disulfide bond, a hydrocarbyldithiomethyl group, and a 2-nitrobenzyloxy group.

In some embodiments, wherein the labeling reagent comprise a plurality of optical moiety attachment sites and wherein the labeled substrate further comprise a plurality of optical moieties attached to the plurality of optical moiety attachment sites.

In another aspect, provided is a sequencing reagent, comprising: a nucleotide; a linker; a fluorescent dye moiety; and a dendrimer comprising a plurality of dendrimer branches each comprising a repeating number of polyethylene glycol (PEG) or hydroxyproline, wherein the linker is attached to a nucleobase of the nucleotide and the dendrimer, and wherein (i) two dendrimer branches of the plurality of dendrimer branches are attached to a dendrimer core of the dendrimer and the fluorescent dye moiety is disposed between the two dendrimer branches and attached to the dendrimer core or (ii) two nth order dendrimer branches of the plurality of dendrimer branches are attached to a (n−1)th dendrimer branch of the dendrimer and the fluorescent dye moiety is disposed between the two nth order dendrimer branches and attached to the (n−1)th dendrimer branch.

In some embodiments, the two dendrimer branches or two nth order dendrimer branches are terminated by a water soluble group.

In some embodiments, the water soluble group comprises sulfonic acid.

In some embodiments, the sequencing reagent of any of claims 51-53, wherein the sequencing reagent comprises at least two fluorescent dye moieties, wherein each respective fluorescent dye of the at least two fluorescent dye moieties (i) is disposed between a respective pair of dendrimer branches, wherein each respective pair of dendrimer branches and each respective fluorescent dye are attached to the dendrimer core or (ii) is disposed between a respective pair of nth order dendrimer branches, wherein each respective pair of nth order dendrimer branches and each respective fluorescent dye are attached to a respective (n−1)th dendrimer branch of the dendrimer.

In some embodiments, the sequencing reagent comprises at least four fluorescent dye moieties.

In some embodiments, the sequencing reagent comprises at least eight fluorescent dye moieties.

In another aspect, provided is a sequencing reagent, comprising: a nucleotide; a linker; a fluorescent dye moiety; and a dendrimer comprising a plurality of dendrimer branches each comprising a repeating number of a polymer, polyethylene glycol (PEG), or hydroxyproline, wherein the linker is attached to a nucleobase of the nucleotide and the dendrimer, and wherein the fluorescent dye moiety is attached to a distal end of a highest order dendrimer branch of the plurality of dendrimer branches relative to a dendrimer core of the dendrimer.

In some embodiments, the sequencing reagent comprises at least two fluorescent dye moieties, wherein each respective fluorescent dye of the at least two fluorescent dye moieties is attached to a respective distal end of a respective highest order dendrimer branch of the plurality of dendrimer branches relative to the dendrimer core.

In some embodiments, the sequencing reagent comprises at least four fluorescent dye moieties.

In some embodiments, the sequencing reagent comprises at least eight fluorescent dye moieties.

In another aspect, provided is a composition, comprising: a sequencing reagent, comprising: a template nucleic acid molecule; a linker; and a dendrimer comprising a plurality of dendrimer branches each comprising a repeating number of a polymer, polyethylene glycol (PEG), or hydroxyproline, wherein the linker is attached to the template nucleic acid molecule and the dendrimer.

In some embodiments, the dendrimer has at least four orders of dendrimer branches.

In some embodiments, the dendrimer has at least six orders of dendrimer branches.

In some embodiments, the dendrimer has at least eight orders of dendrimer branches.

In some embodiments, the dendrimer has at least ten orders of dendrimer branches.

In some embodiments, the highest order dendrimer branches of the plurality of dendrimer branches are terminated by a water soluble group.

In some embodiments, the water soluble group comprises sulfonic acid.

In some embodiments, the sequencing reagent is in solution and not immobilized to a substrate bigger than 1 mm² in surface area.

In some embodiments, the sequencing reagent is immobilized to a substrate bigger than 1 mm² in surface area.

In some embodiments, the sequencing reagent is immobilized to a substrate bigger than 1000 mm² in surface area.

In another aspect, provided is a method, comprising: using the labeling reagent of any one of the above aspects and embodiments in a sequencing reaction comprising providing the labeling reagent to a template nucleic acid molecule hybridized to an extending sequencing primer molecule, wherein the linker of the labeling reagent is coupled to a nucleobase of a nucleotide substrate.

In some embodiments, the method further comprises incorporating the nucleotide substrate in the extending sequencing primer molecule and detecting the fluorescent dye moiety.

In another aspect, provided is a method, comprising: using the sequencing reagent of any one of the above aspects and embodiments in a sequencing reaction comprising providing the sequencing reagent to a template nucleic acid molecule hybridized to an extending sequencing primer molecule.

In some embodiments, the method further comprises incorporating the nucleotide in the extending sequencing primer molecule and detecting the fluorescent dye moiety.

In another aspect, provided is a method for spatially separating objects on a substrate, comprising: loading and immobilizing a plurality of dendrimers as a layer on the substrate, wherein a first dendrimer of the plurality of dendrimers is attached to a nucleic acid template molecule, and wherein the first dendrimer comprises a plurality of dendrimeric branches that spaces the nucleic acid template molecule from an additional nucleic acid template molecule attached to a second dendrimer immobilized adjacent to the dendrimer.

In some embodiments, the method further comprises sequencing the template nucleic acid molecule.

In some embodiments, the sequencing comprises extending a sequencing primer hybridized to the nucleic acid template molecule by providing nucleotide reagents, washing away unincorporated nucleotides from a reaction space, and detecting signals indicative of incorporation, or lack thereof, of the nucleotide reagents.

In another aspect, provided is a method for spatially separating objects on a substrate, comprising: loading and immobilizing a plurality of dendrimers attached to a plurality of binding agents as a layer on the substrate, wherein a first dendrimer of the plurality of dendrimers is attached to a binding agent, and wherein the first dendrimer comprises a plurality of dendrimeric branches that spaces the binding agent from an additional binding agent attached to a second dendrimer immobilized adjacent to the dendrimer; and contacting the plurality of dendrimers immobilized to the substrate with a plurality of template nucleic acid molecules, thereby binding template nucleic acid molecules of the plurality of template nucleic acid molecules to the plurality of binding agents.

In some embodiments, the method further comprises sequencing the template nucleic acid molecule.

In some embodiments, the sequencing comprises extending a sequencing primer hybridized to the nucleic acid template molecule by providing nucleotide reagents, washing away unincorporated nucleotides from a reaction space, and detecting signals indicative of incorporation, or lack thereof, of the nucleotide reagents.

Another aspect of the present disclosure provides a system comprising one or more computer processors and computer memory coupled thereto. The computer memory comprises machine executable code that, upon execution by the one or more computer processors, implements any of the methods above or elsewhere herein. Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 shows components that may be used to construct labelling reagents and labeled substrates.

FIG. 2 shows additional components that may be used to construct labelling reagents and labeled substrates.

FIG. 3 illustrates example labelled substrates comprising a PEG linker portion.

FIG. 4 illustrates example labelled substrates comprising a modified PEG linker portion.

FIG. 5A illustrates an example dendrimeric labelled substrate structure.

FIG. 5B illustrates an example dendrimeric labelled substrate comprising a dUTP substrate.

FIG. 5C illustrates an additional example dendrimeric labelled substrate comprising a dUTP substrate.

FIG. 6A illustrates another example dendrimeric labelled substrate structure.

FIG. 6B illustrates another example dendrimeric labelled substrate comprising a dUTP substrate.

FIG. 6C illustrates an example dendrimer labeled with 8 dye moieties.

FIG. 7 illustrates an example multi-layer dendrimer structure.

FIG. 8 illustrates an example labeled substrate structure comprising a linker comprising a labeled protein.

FIG. 9 illustrates graphs of fluorescence vs time in accordance with an incorporation assay for SUMO1-labeled dUTP.

FIG. 10 illustrates the hydroxyl scar on a dNTP substrate upon cleavage of a linker.

FIG. 11 illustrates experimental results of testing incorporation of streptavidin-labeled substrates.

FIG. 12 illustrates experimental results of testing incorporation of streptavidin-labeled substrates with different length PEG linkers.

FIG. 13 illustrates the intensity vs wavelength plot for various labelled streptavidin scaffolds.

FIGS. 14A-B illustrate the relative fluorescence intensity (FI) measured for linkers and substrates labeled with different number of dyes.

FIG. 15 illustrates a protected fluorophore.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle. A reversible coupling may comprise, for example, a releasable coupling (e.g., in which a first object may be released from a second object to which it is coupled). A first object releasably coupled to a second object may be separated from the second object, e.g., upon application of a stimulus, which stimulus may comprise a photostimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. A coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-π interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. A coupling between a first object and a second object may comprise a labile moiety, such as a moiety comprising an ester, vicinal diol, phosphodiester, peptidic, glycosidic, sulfone, Diels-Alder, or similar linkage. The strength of a coupling between a first object and a second object may be indicated by a dissociation constant, Kd, that indicates the inclination of a coupled object comprising a first object and a second object to dissociate into the uncoupled first and second objects and may be expressed as a ratio of dissociated (e.g., uncoupled) objects to coupled objects. A smaller dissociation constant is generally indicative of a stronger coupling between coupled objects. Coupled objects and their corresponding uncoupled components may exist in dynamic equilibrium with one another. For example, a solution comprising a plurality of coupled objects each comprising a first object and a second object may also include a plurality of first objects and a plurality of second objects. At a given point in time, a given first object and a given second object may be coupled to one another or the objects may be uncoupled; the relative concentrations of coupled and uncoupled components throughout the solution can depend upon the strength of the coupling between the first and second objects (reflected in the dissociation constant).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3) w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The term “terminator” as used herein with respect to a nucleotide may generally refer to a moiety that is capable of terminating primer extension. A terminator may be a reversible terminator. A reversible terminator may comprise a blocking or capping group that is attached to the 3′-oxygen atom of a sugar moiety (e.g., a pentose) of a nucleotide or nucleotide analog. Such moieties are referred to as 3′-O-blocked reversible terminators. Examples of 3′-O-blocked reversible terminators include, for example, 3′-ONH₂ reversible terminators, 3′-O-allyl reversible terminators, and 3′-O-aziomethyl reversible terminators. Alternatively, a reversible terminator may comprise a blocking group in a linker (e.g., a cleavable linker) and/or dye moiety of a nucleotide analog. 3′-unblocked reversible terminators may be attached to both the base of the nucleotide analog as well as a fluorescing group (e.g., label, as described herein). Examples of 3′-unblocked reversible terminators include, for example, the “virtual terminator” developed by Helicos BioSciences Corp, and the “lightning terminator” developed by Michael L. Metzker et al. Cleavage of a reversible terminator may be achieved by, for example, irradiating a nucleic acid molecule including the reversible terminator.

The term “sequencing,” as used herein, generally refers to a process for generating or identifying a sequence of a biological molecule, such as a nucleic acid. The sequence may be a nucleic acid sequence which comprises a sequence of nucleic acid bases. Examples of sequencing include single molecule sequencing or sequencing by synthesis, for example. Sequencing may comprise generating sequencing signals and/or sequencing reads.

The term “context,” as used herein, generally refers to the sequence of the neighboring nucleotides, or context, has been observed to affect the tolerance in an incorporation reaction. The nature of the enzyme, the pH, and other factors may also affect the tolerance. Reducing context effects to a minimum greatly simplifies base determination.

The term “scar,” as used herein, generally refers to a residue left on a previously labeled nucleotide or nucleotide analog after cleavage of an optical (e.g., fluorescent) dye and, optionally, all or a portion of a linker attaching the optical dye to the nucleotide or nucleotide analog. Examples of scars include, but are not limited to, hydroxyl moieties (e.g., resulting from cleavage of an azidomethyl group, hydrocarbyldithiomethyl linkage, or 2-nitrobenzyloxy linkage), thiol moieties (e.g., resulting from cleavage of a disulfide linkage), propargyl moieties (e.g., propargyl alcohol, propargyl amine, or propargyl thiol), and benzyl moieties. For example, a scar may comprise an aromatic group such as a phenyl or benzyl group. The size and nature of a scar may affect subsequent incorporations.

Compounds and chemical moieties described herein, including linkers, may contain one or more asymmetric centers and thus give rise to enantiomers, diastereomers, and other stereoisomeric forms that are defined, in terms of absolute stereochemistry, as (R)- or (S)-, and, in terms of relative stereochemistry, as (D)- or (L)-. The D/L system relates molecules to the chiral molecule glyceraldehyde and is commonly used to describe biological molecules including amino acids. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a phenyl ring. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions,” John Wiley and Sons, Inc., 1981, herein incorporated by reference for this disclosure). Stereoisomers may also be obtained by stereoselective synthesis.

Compounds and chemical moieties described herein, including linkers, may exist as tautomers. A “tautomer” refers to a molecule wherein a proton shift from one atom of a molecule to another atom of the same molecule is possible. In circumstances where tautomerization is possible, a chemical equilibrium of the tautomers may exist. Unless otherwise stated, chemical structures depicted herein are intended to include structures which are different tautomers of the structures depicted. For example, the chemical structure depicted with an enol moiety also includes the keto tautomer form of the enol moiety. The exact ratio of the tautomers depends on several factors, including physical state, temperature, solvent, and pH.

Compounds and chemical moieties described herein, including linkers and dyes, may be provided in different enriched isotopic forms. For example, compounds may be enriched in the content of ²H, ³H, ¹¹C, ¹³C and/or ¹⁴C. For example, a linker, substrate (e.g., nucleotide or nucleotide analog), or dye may be deuterated in at least one position. In some examples, a linker, substrate (e.g., nucleotide or nucleotide analog), or dye may be fully deuterated. Such deuterated forms can be made by the procedure described in U.S. Pat. Nos. 5,846,514 and 6,334,997, each of which are herein incorporated by reference in their entireties. As described in U.S. Pat. Nos. 5,846,514 and 6,334,997, deuteration can improve the metabolic stability and or efficacy, thus increasing the duration of action of drugs. Unless otherwise stated, structures depicted and described herein are intended to include compounds which differ only in the presence of one or more isotopically enriched atoms. For example, compounds and chemical moieties having the present structures except for the replacement of a hydrogen by a deuterium or tritium, or the replacement of a carbon by ¹³C- or ¹⁴C-enriched carbon are within the scope of the present disclosure. The compounds and chemical moieties of the present disclosure may contain unnatural proportions of atomic isotopes at one or more atoms that constitute such compounds. For example, a compound or chemical moiety such as a linker, substrate (e.g., nucleotide or nucleotide analog), or dye, or a combination thereof, may be labeled with one or more isotopes, such as deuterium (²H), tritium (³H), iodine-¹²⁵ (¹²⁵I) or carbon-¹⁴ (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds and chemical moieties as described herein whether radioactive or not, are encompassed within the scope of the present disclosure.

The term “analyte,” as used herein, generally refers to an object that is the subject of analysis, or an object, regardless of being the subject of analysis, that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

As used herein, the term “template nucleic acid” generally refers to the nucleic acid to be sequenced. The template nucleic acid may be an analyte or be associated with an analyte. For example, the analyte can be a mRNA, and the template nucleic acid is the mRNA or a cDNA derived from the mRNA, or other derivative thereof. In another example, the analyte can be a protein, and the template nucleic acid is an oligonucleotide that is conjugated to an antibody that binds to the protein, or derivative thereof. Examples of sequencing include single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

### Labelling Components

A substrate may be labeled with an optical moiety, such as a dye moiety. The optical moiety may be attached to the substrate via a linker. Thus, a labeled substrate may comprise a linker and an optical moiety. In some cases, a substrate may be labeled with a labelling reagent comprising a linker and an optical moiety. In some cases, a labeled substrate may be or comprise the labelling reagent. Labeled substrates may be detected, such as in an imaging operation. The imaging operation may comprise exciting the optical moiety (e.g., dye) using light provided at a first wavelength(s) and detecting light at a second wavelength(s).

A labeled substrate may be used to optically probe an analyte, for example by providing the labeled substrate to couple to or react with the analyte and detecting one or more signals deriving from the labeled substrate or reaction therefrom. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically cleavable (e.g., via a reducing agent, such as dithiothreitol (DTT), tris(2-carboxyethyl)phosphine (TCEP), tris(hydroxypropyl)phosphine (THP) or enzymatically cleavable (e.g., via an esterase, lipase, peptidase or protease). The probe may detect the presence or absence of the analyte. The probe may detect the presence or absence of a characteristic or parameter of the analyte that relates to the probe. In an example, the substrate is a nucleotide, and labeled nucleotides are used to probe a template nucleic acid in order to sequence the template nucleic acid in a sequencing operation (e.g., single molecule sequencing, sequencing by synthesis, sequencing by ligation, sequencing by binding, etc.). In another example, the substrate is an oligonucleotide, and labeled oligonucleotides are used to probe a sample in order to determine the presence or absence of a gene sequence in the sample. In another example, the substrate is an antibody or oligonucleotide-conjugated antibody, and labeled antibodies or labeled oligonucleotide-conjugated antibodies are used to probe a sample to determine the presence or absence of a protein in the sample. The substrate may comprise any molecule or molecules that can be labeled by the components and mechanisms described herein. The substrate can be any suitable molecule, analyte, cell, tissue, or surface that is to be optically labeled. Examples include cells, including eukaryotic cells, prokaryotic cells, healthy cells, and diseased cells; cellular receptors; antibodies; proteins; lipids; metabolites; saccharides; polysaccharides; probes; reagents; nucleotides and nucleotide analogs (e.g., as described herein); polynucleotides; and nucleic acid molecules.

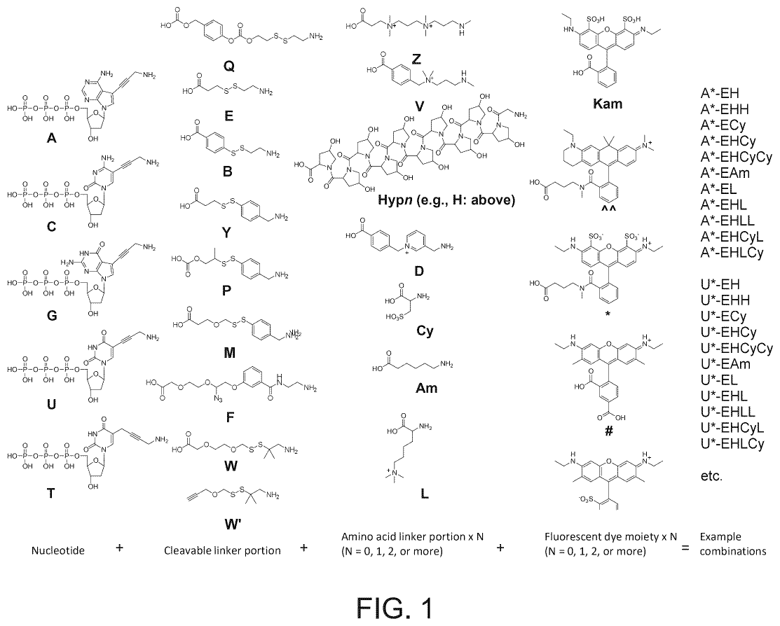

FIG. 1 shows a variety of components that may be used in the construction of labelling reagents and labeled substrates. A linker between the substrate and the optical moiety may comprise one or more of a cleavable linker moiety, a semi-rigid linker moiety, an amino acid, multiples thereof, or any combination thereof. FIG. 1 illustrates example nucleotide substrates, propargylamino functionalized nucleotides (A, C, G, T, and U), but any other useful nucleotide or nucleotide analog with any other useful chemical handle can be used. Non-nucleotide substrates may be labeled using the component(s) shown in FIG. 1. Cleavable linker moieties include, for example, the structures shown as: Q, E, B, Y, P, M, F, W, and W′. A cleavable linker moiety may include a cleavable group as described herein. For example, some of the listed cleavable linker moieties include disulfide bonds. A semi-rigid linker moiety may comprise one or more amino acid moieties, including, for example, one or more hydroxyproline moieties as described herein. For example, a linker may comprise a hydroxyproline linker (Hypₙ). The “H” linker moiety illustrated in FIG. 1 is a hyp10 moiety. The hydroxyproline linker (Hypₙ) may comprise any useful number of hydroxyproline residues (e.g., Hyp3, Hyp6, Hyp9, Hyp10, Hyp20, Hyp30, Hyp40, etc.) and, in some cases, another moiety such as a glycine moiety, as described herein. In some cases, a group of consecutive hydroxyproline residues may be separated by one or more other moieties or features (e.g., [Hyp10]-[another moiety]-[Hyp10]). The amino acid may comprise cysteic acid (e.g., the “Cy” moiety), 5-amino-5-carboxy-N,N,N-trimethylpentan-1-aminium or a salt thereof (e.g., the “L” moiety), 6-aminohexanoic acid (e.g., the “Am” moiety), “C” moiety, a quaternary amine (e.g., the “V” moiety or “Z” moiety), multiples thereof, or any combination thereof. A linker may include multiple portions including multiple different amino acids in any order. An optical moiety may be a fluorescent dye moiety such as the structures of “Kam”, “*,” “#,” “$,” “\hat{ }\hat{ }.” or any other useful structure, such as any of the dyes or labels described elsewhere herein. Throughout the application, wherever such labels are used, any other optical moiety may be substituted. In some cases, a dye may be represented as “‡,” which symbol is intended to represent any useful dye moiety or combination of dye moieties (e.g., dye pairs). In some cases, a dye may be red-fluorescing or green-fluorescing.

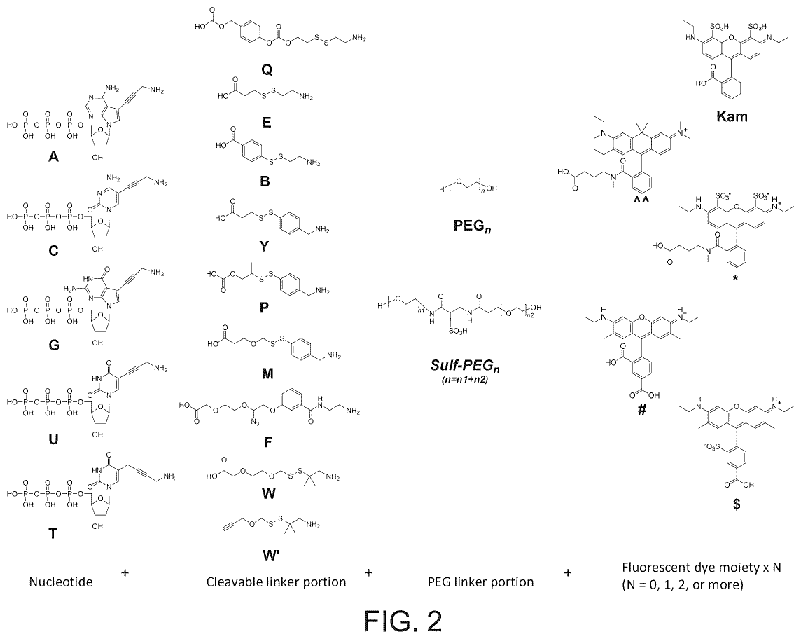

FIG. 2 shows a variety of additional components in the linker, such as the “PEGₙ” and “Sulf-PEG,”, that may be used in the construction of labelling reagents and labeled substrates. For example, a linker between the substrate (described with respect to FIG. 1) and the optical moiety (described with respect to FIG. 1) may comprise one or more of a cleavable linker moiety, a polyethylene glycol (PEG) linker moiety, a modified PEG linker moiety, a semi-rigid linker moiety, an amino acid, multiples thereof, or any combination thereof. A linker may include multiple portions including multiple different amino acids in any order. In some cases, a linker may comprise linker component(s) that carry the same or similar charge as that of the dye moiety connected to the linker (e.g., at biological pH). For example, a linker attached to the KAM or * dye may comprise linker component(s) comprising sulfonic acid and/or carboxylic acid components. In another example, a linker attached to the \hat{ }\hat{ } dye may comprise linker component(s) comprising a quaternary ammonium. In some cases, a linker may comprise only linker component(s) that carry the same or similar charge as that of the dye moiety connected to the linker (e.g., at biological pH).

A labeled substrate may comprise any number of linkers and any number of optical moieties. A linker may each be attached to one optical moiety (e.g., dye moiety) or multiple optical moieties (e.g., dye moieties). In some cases, multiple optical moieties on a same linker or labeled substrate may be detectable at a single wavelength or wavelength range. In some cases, multiple optical moieties on a same linker or labeled substrate may be detected at different wavelength or wavelength range. In some cases, a labeled substrate may comprise a branched or dendritic structure (e.g., as described herein) comprising multiple linker moieties (e.g., multiple sets of hydroxyproline moieties connected at different branch points to a central structure), which linker moieties may be the same or different. In some cases, a labeled substrate may comprise multiple dyes attached to different locations of a linker (e.g., different locations throughout a hydroxyproline moiety). In some cases, a labeled substrate may comprise multiple optical moieties wherein at least one is a quencher. A linker may comprise any combination of ‘cleavable linker portion’, ‘amino acid linker portion’, and ‘PEG linker portion’ components illustrated in FIGS. 1-2, including multiples thereof in any order. A labeled substrate may comprise any combination of ‘cleavable linker portion’, ‘amino acid linker portion’, and ‘PEG linker portion’ components illustrated in FIGS. 1-2, including multiples thereof in any order. There are numerous possible variations of linkers and labeled substrates that may be constructed using various permutations of the components illustrated in FIGS. 1-2, appreciating that the various linker components can be ordered in any number, any order, and in combination with or without additional moieties (e.g., such as a glycine moiety) disposed at various locations. Labeled substrates may be prepared according to synthetic routes and principles described herein. Provided herein are also unlabeled substrates. Provided herein are also mixtures of labeled and unlabeled substrates (e.g., a mixture of labeled and unlabeled nucleotides). In some cases, the substrate is a nucleotide. Any natural nucleotide, modified nucleotide, or nucleotide analog may be the substrate, such as a reversibly terminated nucleotide or unterminated nucleotide. Various linkers, labeling reagents, labels, substrates, and combinations thereof are described in further detail in U.S. Pat. No. 11,377,680B2, International Patent Pub. No. WO2022/040213A1, International Patent Pub. No. WO2023/023357A2, International Patent Pub. No. WO2023/164003A2, and International Patent. App. No. PCT/US2024/018563, each of which is entirely incorporated by reference herein for all purposes.

### Optical Moieties

An optical moiety may also be referred to herein as a “label.” An optical moiety generally refers to a detectable moiety that emits a signal (or reduces an already emitted signal) that can be detected. The label may be luminescent (e.g., fluorescent or phosphorescent). For example, the label may be or comprise a fluorescent moiety (e.g., a dye). Non-limiting examples of dyes include SYBR green, SYBR blue, DAPI, propidium iodine, Hoechst, SYBR gold, ethidium bromide, acridine, proflavine, acridine orange, acriflavine, fluorocoumarin, ellipticine, daunomycin, chloroquine, distamycin D, chromomycin, homidium, mithramycin, ruthenium polypyridyls, anthramycin, phenanthridines and acridines, propidium iodide, hexidium iodide, dihydroethidium, ethidium homodimer-1 and -2, ethidium monoazide, ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO labels (e.g., SYTO-40, -41, -42, -43, -44, and -45 (blue); SYTO-13, -16, -24, -21, -23, -12, -11, -20, -22, -15, -14, and -25 (green); SYTO-81, -80, -82, -83, -84, and -85 (orange); and SYTO-64, -17, -59, -61, -62, -60, and -63 (red)), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5-(or 6-) iodoacetamidofluorescein, 5-{[2 (and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor labels (e.g., AlexaFluor 350, 405, 430, 488, 532, 546, 555, 568, 594, 610, 633, 635, 647, 660, 680, 700, 750, and 790 dyes), DyLight labels (e.g., DyLight 350, 405, 488, 550, 594, 633, 650, 680, 755, and 800 dyes), Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), ATTO fluorescent quenchers (ATTO-TEC GmbH) (e.g., ATTO 540Q, ATTO 580Q, ATTO 612Q, Atto532 [e.g., ATTO 532 succinimidyl ester], and Atto633), Kam, and other fluorophores and/or quenchers. A fluorescent dye may be excited by application of energy corresponding to the visible region of the electromagnetic spectrum (e.g., between about 430-770 nanometers (nm)). Excitation may be done using any useful apparatus, such as a laser and/or light emitting diode. A fluorescent dye may emit light (e.g., fluoresce) in the visible region of the electromagnetic spectrum ((e.g., between about 430-770 nm). A fluorescent dye may be excited over a single wavelength or a range of wavelengths. A fluorescent dye may be excitable by light in the red region of the visible portion of the electromagnetic spectrum (about 625-740 nm) (e.g., have an excitation maximum in the red region of the visible portion of the electromagnetic spectrum). Alternatively or additionally, fluorescent dye may be excitable by light in the green region of the visible portion of the electromagnetic spectrum (about 500-565 nm) (e.g., have an excitation maximum in the green region of the visible portion of the electromagnetic spectrum). A fluorescent dye may emit signal in the red region of the visible portion of the electromagnetic spectrum (about 625-740 nm) (e.g., have an emission maximum in the red region of the visible portion of the electromagnetic spectrum). Alternatively or additionally, fluorescent dye may emit signal in the green region of the visible portion of the electromagnetic spectrum (about 500-565 nm) (e.g., have an emission maximum in the green region of the visible portion of the electromagnetic spectrum). A label may be a quencher. The term “quencher,” as used herein, generally refers to molecules that may be energy acceptors. A quencher may be a molecule that can reduce an emitted signal. Luminescence from labels may also be quenched. In some cases, the label may be a type that does not self-quench or exhibit proximity quenching. Non-limiting examples of a label type that does not self-quench or exhibit proximity quenching include Bimane derivatives such as Monobromobimane. The term “proximity quenching,” as used herein, generally refers to a phenomenon where one or more dyes near each other may exhibit lower fluorescence as compared to the fluorescence they exhibit individually. In some cases, the dye may be subject to proximity quenching wherein the donor dye and acceptor dye are within 1 nm to 50 nm of each other. Examples of quenchers include, but are not limited to, Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), and ATTO fluorescent quenchers (ATTO-TEC GmbH) (e.g., ATTO 540Q, ATTO 580Q, and ATTO 612Q). Fluorophore donor molecules may be used in conjunction with a quencher. Examples of fluorophore donor molecules that can be used in conjunction with quenchers include, but are not limited to, fluorophores such as Cy3B, Cy3, or Cy5; Dy-Quenchers (Dyomics) (e.g., DYQ-660 and DYQ-661); and ATTO fluorescent quenchers (ATTO-TEC GmbH) (e.g., ATTO 540Q, 580Q, and 612Q). A labeling reagent described herein may have at least, at most, or about 1, 2, 3, 4, 5, 6, 7, 8, 9, or 10 fluorescent dye moieties.

### Linkers

An association between a linker and a substrate can be any suitable association including a covalent or non-covalent bond. For example, a linker may be coupled to a substrate (e.g., nucleotide) via a nucleobase of a nucleotide via, e.g., a propargyl or propargylamino moiety. In another example, a linker may be coupled to a substrate (e.g., protein, such as an antibody) via an amino acid of a polypeptide or protein. In some cases, an association between a linker and a substrate may be a biotin-avidin interaction. In other cases, an association between a linker and a substrate may be via a propargylamino moiety. In some cases, an association between a linker and a substrate may be via an amide bond (e.g., a peptide bond). A linker may comprise a cleavable moiety configured to be cleaved to separate the labeling reagent or a portion thereof from a substrate to which it is attached.

A linker may comprise an amino acid. A linker may comprise at least and/or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80 amino acids. A linker may comprise a plurality of different types of amino acids. An amino acid may be proteinogenic or non-proteinogenic. A “proteinogenic amino acid,” as used herein, generally refers to a genetically encoded amino acid that may be incorporated into a protein during translation. Proteinogenic amino acids include arginine, histidine, lysine, aspartic acid, glutamic acid, serine, threonine, asparagine, glutamine, cysteine, selenocysteine, glycine, proline, alanine, isoleucine, leucine, methionine, phenylalanine, tryptophan, tyrosine, valine, selenocysteine, and pyrrolysine. A “non-proteinogenic amino acid,” as used herein, is an amino acid that is not a proteinogenic amino acid. A non-proteinogenic amino acid may be a naturally occurring amino acid or a non-naturally occurring amino acid. Non-proteinogenic amino acids include amino acids that are not found in proteins and/or are not naturally encoded or found in the genetic code of an organism. Examples of non-proteinogenic amino acid include, but are not limited to, (all-S,all-E)-3-amino-9-methoxy-2,6,8-trimethyl-10-phenyldeca-4,6-dienoic acid (ADDA), 2-aminoisobutyric acid, ay-aminobutyric acid, 4-aminobenzoic acid, 4-hydroxyphenylglycine, 6-aminohexanoic acid, aminolevulinic acid, 8-aminolevulinic acid, azetidine-2-carboxylic acid, alloisoleucine, allothreonine, canaline, canavanine, carboxyglutamic acid, chloroalanine, citrulline, cysteic acid, 5-amino-5-carboxy-N,N,N-trimethylpentan-1-aminium (also known as 2-amino-itr6-(trimethylammonio) hexanoate), dehydroalanine, diaminopimelic acid, dihydroxyphenylglycine, enduracididine, gamma-aminobutyric acid, hawkinsin, homocysteine, homoserine, hydroxyproline, hypusine, isovaline, isoserine, lanthionine, t-leucine, norleucine, norvaline, nv-5138, ornithine, penicillamine, pipecolic acid, plakohypaphorine, pyroglutamic acid, quisqualic acid, s-aminoethyl-1-cysteine, sarcosine, theanine, tranexamic acid, tricholomic acid, β-alanine (3-aminopropanoic acid), or β-leucine, selenomethionine, α-amino-n-heptanoic acid, α,β-diaminopropionic acid, α,γ-diaminobutyric acid, β-amino-n-butyric acid, β-aminoisobutyric acid, N-ethyl glycine, N-propyl glycine, N-isopropyl glycine, N-methyl alanine, N-ethyl alanine, N-methyl β-alanine, N-ethyl β-alanine, α-hydroxy-γ-aminobutyric acid, trans-4-aminomethylcyclohexane carboxylic acid, and 4-hydrazinobenzoic acid. A non-proteinogenic amino acid may comprise a ring structure. A non-proteinogenic amino acid may be aliphatic, branched, or cyclic. A non-proteinogenic amino acid may be non-cyclic. A non-proteinogenic amino acid may be positively charged, for example, carry at least 1, 2, 3, 4, 5, or more positive charges. A non-proteinogenic amino acid may be negatively charged, for example, carry at least 1, 2, 3, 4, 5, or more negative charges. A non-proteinogenic amino acid may also be neutral or not carry a charge. A non-proteinogenic amino acid may comprise a side-chain chemical moiety, for example, at least 1, 2, 3, 4, 5, or more side chain chemical moieties. A linker may comprise a proteinogenic amino acid. A linker may comprise a non-proteinogenic amino acid. A linker may comprise at least and/or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80 or more proteinogenic amino acids. A linker may comprise at least and/or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80 or more non-proteinogenic amino acids. Where a linker comprises multiple amino acids, such as multiple non-proteinogenic amino acids, an amine moiety adjacent to a ring moiety (e.g., the amine moiety in the hydrazine moiety) can function as a water-solubilizing group. Other moieties can be used to increase water-solubility, such as by linking amino acids with oxamate moieties.

A linker may comprise a quaternary amine. For example, a linker may comprise at least and/or at most about 1, 2, 3, 4, 5, 6, 7, 8, 9, 10 or more quaternary amine subunits. Where multiple quaternary amine subunits are present, in some cases, they may be linked consecutively, or one or more quaternary amine subunits may be separated by other linker subunits (e.g., amino acid subunits, e.g., Hypₙ).

A linker may comprise a semi-rigid portion. The semi-rigid portion of the linker may provide physical separation between the substrate and the optical moiety, which physical separation may facilitate, e.g., effective labeling of the substrate with the labeling reagent, effective detection of the labeling reagent coupled to the substrate, effective labeling of the substrate with additional labeling reagents (e.g., in the case of incorporation into homopolymeric regions of a nucleic acid template), etc. For example, the semi-rigid portion may provide physical separation of, on average, at least 9 Å, 12 Å, 15 Å, 18 Å, 21 Å, 24 Å, 27 Å, 30 Å, 33 Å, 36 Å, 39 Å, 42 Å, 45 Å, 48 Å, 51 Å, 54 Å, 57 Å, 60 Å, 63 Å, 66 Å, 69 Å, 72 Å, 75 Å, 78 Å, 81 Å, 84 Å, 87 Å, 90 Å, or more between the substrate and the optical moiety. This average separation may vary with environmental conditions including, for example, solvents (or lack thereof), temperature, pH, pressure, etc. A semi-rigid portion of a linker may comprise a secondary structure such as a helical structure that establishes and maintains a degree of physical separation between the substrate and the optical moiety. The helical structure can comprise prolines and/or hydroxyprolines (e.g., polyproline or polyhydroxyproline helix). The semi-rigid portion may comprise an amino acid, e.g., non-proteinogenic amino acid. Non-proteinogenic amino acids of a linker may be included in any useful portion of the linker and may be included in sequence or separated by one or more other chemical moieties. A semi-rigid portion of a linker may comprise a series of ring systems (e.g., aliphatic and aromatic rings). As used herein, a ring (e.g., ring structure) is a cyclic moiety comprising any number of atoms connected in a closed, essentially circular fashion, as used in the field of organic chemistry. A linker, or a semi-rigid portion thereof, can have any number of rings, including at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80 or more rings. The rings can share an edge in some cases (e.g., be components of a bicyclic ring system). In general, the ring portion of the linker can provide a degree of physical rigidity to the linker and/or facilitate physical separation between objects attached to the linker. A ring can be a component of an amino acid (e.g., a non-proteinogenic amino acid, as described herein). For example, a linker may comprise a proline moiety or a hydroxyproline moiety. For example, a linker, or a semi-rigid portion thereof, may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80 or more proline or hydroxyproline moieties. In some cases, different portions of the linkers may be separated by one or more moieties such as glycine moieties, e.g., a first hydroxyproline section of the linker may be separated from a second hydroxyproline section of the linker with a glycine moiety. A linker may comprise one or more water-soluble groups. A linker may include one or more asymmetric (e.g., chiral) centers (e.g., as described herein). All stereochemical isomers of linkers are contemplated, including racemates and enantiomerically pure linkers. A labeling reagent or component thereof, and/or a substrate, may include one or more isotopic (e.g., radio) labels (e.g., as described herein). All isotopic variations of linkers are contemplated.

A labeling reagent or linker can establish any suitable functional distance between an optical moiety and a substrate, such as at least and/or at most about 500 nanometers (nm), about 200 nm, about 100 nm, about 75 nm, about 50 nm, about 40 nm, about 30 nm, about 20 nm, about 10 nm, about 5 nm, about 2 nm, about 1.0 nm, about 0.5 nm, about 0.3 nm, or about 0.2 nm. In some instances, the functional length is at least and/or at most about 9 Å, 12 Å, 15 Å, 18 Å, 21 Å, 24 Å, 27 Å, 30 Å, 33 Å, 36 Å, 39 Å, 42 Å, 45 Å, 48 Å, 51 Å, 54 Å, 57 Å, 60 Å, 63 Å, 66 Å, 69 Å, 72 Å, 75 Å, 78 Å, 81 Å, 84 Å, 87 Å, 90 Å, or more.

A linker may comprise a polymer (or a polymer linker moiety (ies). A linker may comprise a polymer having a regularly repeating unit. Alternatively, a labeling reagent may comprise a co-polymer without a regularly repeating unit. A repeating unit may comprise a sequence of amino acids (e.g., non-proteinogenic amino acids). A repeating unit may comprise two or more different amino acids. For example, a linker may comprise a moiety having the formula (XₙYₘ)ᵢ, where X is a first amino acid, Y is a second amino acid, n is at least 1, m is at least 1, and i is at least 2, and X and Y are different amino acids. In an example, X may be glycine, n is 1, and Y is hydroxyproline. In such an instance, m may be at least 3 (e.g., 3, 4, 5, 6, 7, 8, 9, 10, 11, 12, 13, 14, 15, 16, 17, 18, 19, 20, or more) and i may be, for example, at least 2 (e.g., 2, 3, 4, 5, 6, 7, 8, or more).

A linker may comprise a PEG polymer having an ethylene oxide as a regularly repeating unit. A linker may comprise a modified PEG polymer, which comprises a PEG polymer and one or more modifying moieties, such as a charged moiety (e.g., positively charged moiety, negatively charged moiety), disposed between repeating units of the PEG polymer. A modifying moiety may be negatively charged (e.g., at biological pH), such as a sulfonic acid moiety, a carboxylic acid moiety, or a phosphate moiety. A modifying moiety may be positively charged (e.g., at biological pH), such as a quaternary amine moiety (e.g., “Z”, “V”, “L” in FIG. 1) or other amine moieties. Thus, a modified PEG polymer may comprise two or more PEG segments with a modifying moiety disposed between pairs of neighboring PEG segments. If there are multiple modifying moieties, the modifying moieties may be the same or different moieties. FIG. 2 illustrates, for example, a PEG linker portion comprising a modified PEG polymer (Sulf-PEGₙ) comprising two PEG segments (PEGₙ₁ and PEGₙ₂), where n1+n2=n, separated by a sulfonic acid moiety (e.g., negatively charged moiety) as the modifying moiety. The PEG or modified PEG may be provided at any useful length, PEGₙ, with any number of repeating units (e.g., ethylene oxide), n. For example, the PEG may have at least about, at most about, and/or about 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400 or more repeating units. A modified PEG polymer may be segmented to any number of segments, such as at least about, at most about, and/or about 1, 2, 3, 4, 5, 6, 7, 8, 9, 10, 11, 12, 13, 14, 15, 16, 17, 18, 19, 20 or more segments, where each segment may have the same or different lengths of repeating PEG units. A PEG segment may have any useful length, with any number of repeating units (e.g., ethylene oxide), for example, at least about, at most about, and/or about 2, 3, 4, 5, 6, 7, 8, 9, 10, 15, 20, 25, 30, 35, 40, 45, 50, 55, 60, 65, 70, 75, 80, 85, 90, 95, 100, or more repeating units. In some cases, the PEG or modified PEG may be provided at any useful molar mass, for example, at least about, at most about, and/or about 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10 k, 10.5 k, 11 k, 11.5 k, 12 k, 12.5 k, 13 k, 13.5 k, 14 k, 14.5 k, 15 k, 15.5 k, 16 k, 16.5 k 17 k, 17.5 k, 18 k, 18.5 k, 19 k, 19.5 k, 20 k or more g/mol or daltons (Da). A modified PEG may comprise a charged modifying moiety that is the same charge as the optical moiety (e.g., dye), such as negatively charged, which beneficially acts to repel each other and rigidify the linker. A modified PEG may comprise two or more charged modifying moieties that carry the same charge, positive or negative, which can repel each other and rigidify the linker.

As used herein, the notation PEGₙ, where n is sub-scripted, generally refers to a PEG polymer having n repeating units, unless explicitly noted or drawn otherwise. As used herein, the notation PEGₙ, where n is not sub-scripted, generally refers to a PEG polymer having n Da of molecular mass or average molecular mass, unless explicitly noted or drawn otherwise.

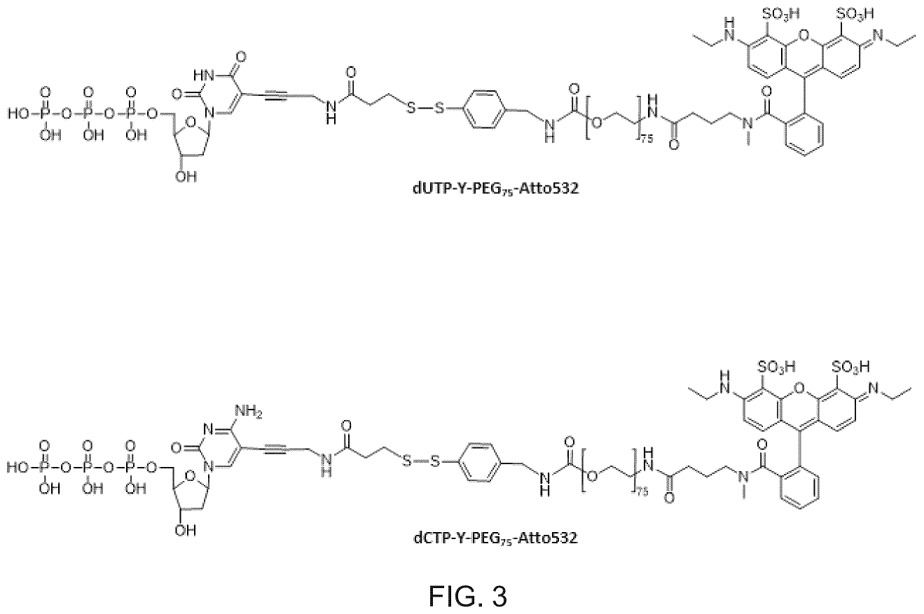

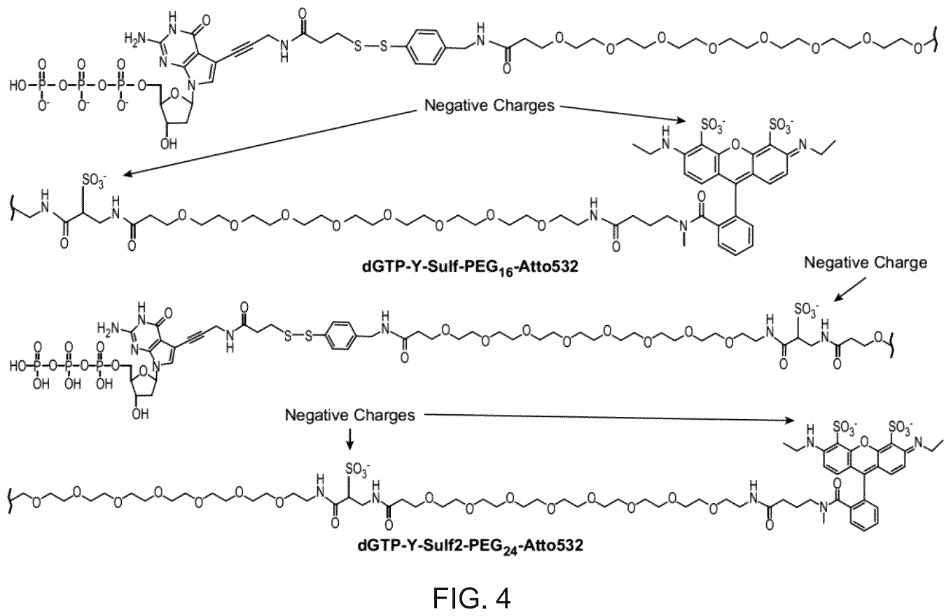

FIG. 3 illustrates example labelled substrates comprising a PEG linker portion. The top component is a dUTP-Y-PEG₇₅-Atto532 and the bottom component is a dCTP-Y-PEG₇₅-Atto532. FIG. 4 illustrates example labelled substrates comprising a modified PEG linker portion. The top component is a dGTP-Y-Sulf-PEG₁₆-Atto532, in which a sulfonic acid moiety is disposed between two PEG₈ segments of the PEG linker. The bottom component is a dGTP-Y-Sulf2-PEG₂₄-Atto532, in which two sulfonic acid moieties are disposed between three PEG₈ segments of the PEG linker. Each of the sulfonic acid moieties and the dye in these components are negatively charged.

Another example linker component is shown below:

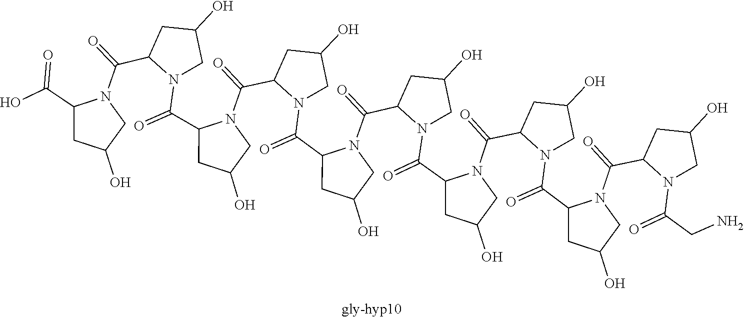 [SMILES: NCC(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)O]

The following labels of: “Hypₙ”, “Hypₙ”, “hypₙ”, “hypₙ”, as used herein, which may generally describe a unit of n hydroxyproline moieties, unless explicitly described otherwise (e.g., “gly-”, “Gly-”, “Gly”-, “gly”-, “with glycine”, “without glycine”, as drawn, etc.) may refer to a structure which may or may not have one or more glycine moieties. For example, such labels may describe a structure of n hydroxyproline moieties with a glycine moiety at an end, a structure of n hydroxyproline moieties which may have one or more glycine moieties between hydroxyprolines, or a structure of n hydroxyproline moieties without any glycine moieties. The structure shown above includes 10 hydroxyproline moieties and a glycine moiety and is referred to herein as “H” “gly-hyp₁₀”, GlyHyp₁₀, Gly-Hyp₁₀, glyhyp10, gly-hyp₁₀, hyp₁₀-gly, or similar. One or more such structures may be included in a labeling reagent or linker portion thereof. For example, a gly-hyp10 structure may be a repeating unit in a linker. Two gly-hyp10 structures in sequence may be referred to herein as hyp20 (having two glycines), or gly-hyp10-gly-hyp10. Such a structure may include 20 hydroxyproline moieties and, in some cases, one or more (e.g., two) glycines. Similarly, three gly-hyp10 structures in sequence may be referred to herein as gly-hyp30. Such a structure may include 30 hydroxyproline moieties and one or more glycines. For example, a gly-hyp30 sequence may include three sets of ten hydroxyprolines separated by glycines. Alternatively, a hyp30 structure may include thirty hydroxyprolines with no intervening structures. Related structures including different numbers of hydroxyprolines (e.g., hypₙ or hypₙ) may also be included in a labeling reagent. As described herein, all stereoisomers of gly-hyp10, gly-hyp20, and hyp30, as well as combinations thereof, are contemplated.

### Cleavable Moieties

A labeling reagent may include one or more cleavable moieties. A cleavable moiety may comprise a cleavable group such as a disulfide moiety. A cleavable moiety may comprise a chemical handle for attachment to a substrate (e.g., as described herein). Accordingly, a cleavable moiety may be included in a labeling reagent at a position adjacent to a substrate to which the labeling reagent is attached. A cleavable moiety may be coupled to a linker component of a labeling reagent via, for example, reaction between a free carboxyl moiety of the linker component and an amino moiety of a cleavable moiety (e.g., cleavable linker portion). A cleavable linker portion may be attached to a substrate upon reaction between a carboxyl moiety of the cleavable linker moiety and an amine moiety attached to a substrate to provide the substrate attached to the cleavable linker portion via an amide moiety.

A cleavable moiety may be cleaved via exposure to one or more stimuli, such as chemical (e.g., reducing agent), heat, enzymatic, light, etc. In some cases, the reducing reagent comprises tetrahydropyran, β-mercaptoethanol (β-ME), dithiothreitol (DTT), tris(2-carboxyethyl)phosphine (TCEP), Ellman's reagent, hydroxylamine, or cyanoborohydride.

FIG. 1 illustrates different examples of cleavable groups that can be a part of a linker, labelled “Q,” “E,” “B,” “Y,” and “P”. A linker may comprise any of these cleavable group examples.

### Dendrimeric Labelling

Provided herein are dendrimeric labelling reagents and dendrimeric labelled substrates.

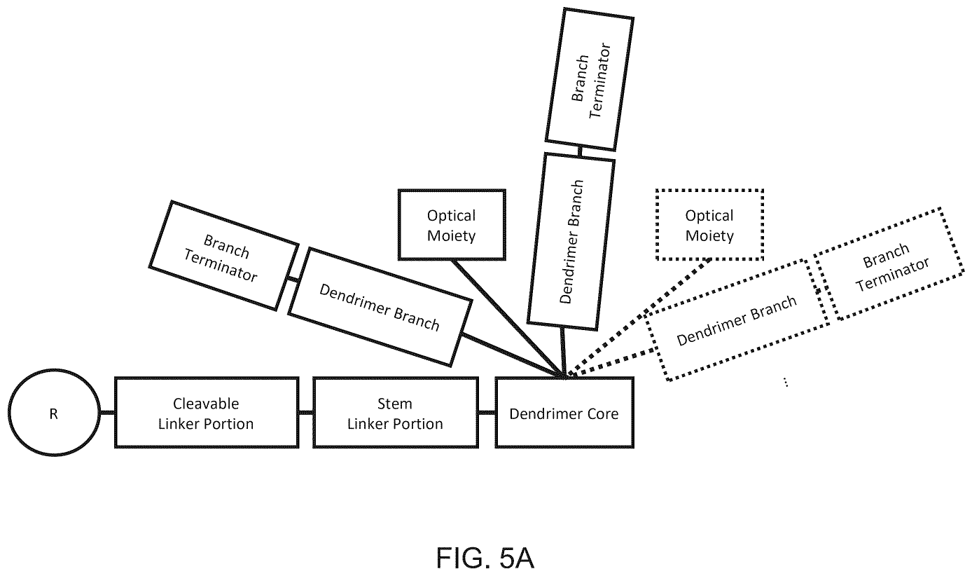

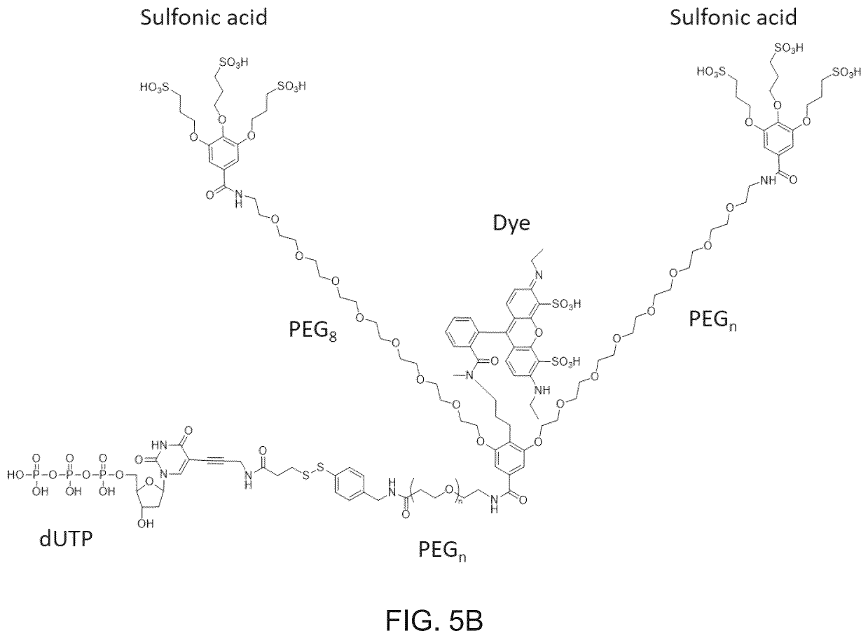

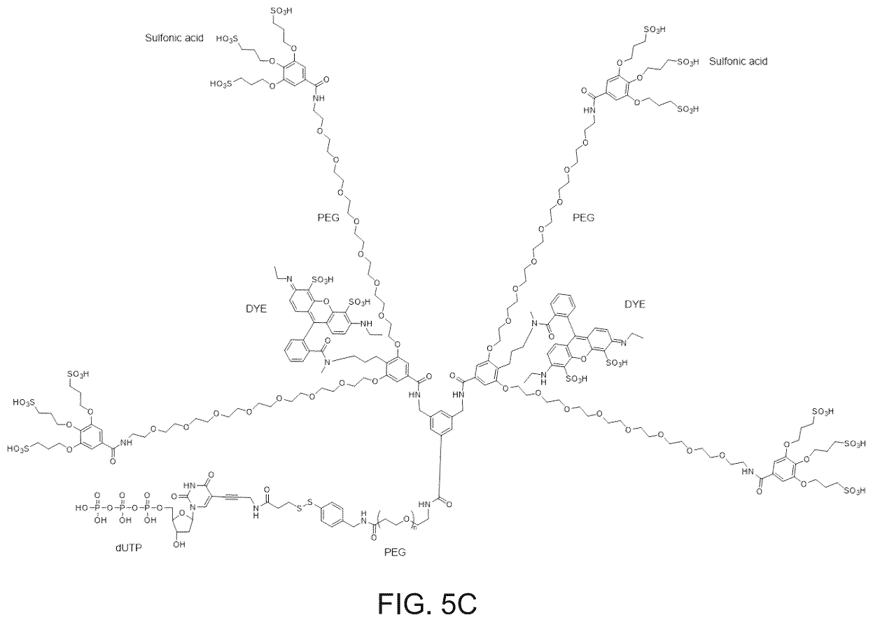

FIG. 5A illustrates an example dendrimeric labelled substrate comprising (i) a substrate “R”, (ii) a linker comprising a cleavable linker portion, a stem linker portion, a dendrimer core, and dendrimeric branches, and (iii) an optical moiety. The dendrimer core may be covalently attached to each dendrimeric branch, optical moiety, and the stem linker portion. A dendrimeric branch may be attached to the dendrimer core as a first order branch, second order branch, third order branch, or higher order branch. For example, second or higher order branches may be attached to the core via lower order branch(es). In the example of FIG. 5A, each dendrimeric branch is a first order branch from the core. Multiple dendrimeric branches attached to the dendrimer core may be of the same order or different order. A dendrimeric branch may comprise a PEG segment of any length, for example, having at least about, at most about, and/or about 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400 or more repeating units. The PEG segment may be provided at any useful molar mass, for example, at least about, at most about, and/or about 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10 k, 10.5 k, 11 k, 11.5 k, 12 k, 12.5 k, 13 k, 13.5 k, 14 k, 14.5 k, 15 k, 15.5 k, 16 k, 16.5 k 17 k, 17.5 k, 18 k, 18.5 k, 19 k, 19.5 k, 20 k or more g/mol or daltons (Da). Alternatively or in addition, a dendrimeric branch may comprise any linker segment of any length or any number of repeating units described elsewhere herein, such as a nonproteinogenic amino acid (e.g., hydroxyproline, Hypₙ). Alternatively or in addition, a dendrimeric branch may comprise any polymer or moiety of any length or any number of repeating units. For example, a dendrimeric branch may comprise a repeating polymer or moiety, having at least about, at most about, and/or about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more repeating units. In another example, a dendrimeric branch may comprise a hydroxyproline segment of any length, having at least about, at most about, and/or about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more repeating units. In some cases, a dendrimeric branch may itself comprise a dendrimer, comprising its own dendrimeric core and dendrimeric branches. Each dendrimeric branch may have the same or different lengths.

The optical moiety may be disposed between at least two dendrimer branches. Beneficially, the at least two dendrimer branches may shield the optical moiety from other optical moieties in the vicinity such as another optical moiety attached to the same labelling reagent or another optical moiety attached to a different labelling reagent. In some cases, the labelling reagent or labeled substrate may comprise multiple optical moieties, each optical moiety being disposed between at least a different pair of dendrimer branches, where the different pair of dendrimer branches may or may not share a common dendrimer branch. Beneficially, the dendrimer branches may shield the different multiple optical moieties from each other. The optical moieties may be the same or different optical moieties. The optical moieties may be the same or different charge. While FIG. 5A illustrates a structure in which an optical moiety is disposed between and shielded by two first order dendrimer branches, with the optical moiety and two first order dendrimer branches being attached to the dendrimer core, it will be appreciated that similarly an optical moiety may be disposed between and shielded by two of any nth order dendrimer branches, with the optical moiety and the nth order dendrimer branches being attached to an (n−1)th order dendrimer branch.

A dendrimer branch may be terminated by a branch terminator group. The branch terminator group may comprise a water-soluble group. The dendrimer branches may be terminated by the same group or different groups. In some cases, each water-soluble group terminating the dendrimer branch may be the same charge, for example each a negative charge or each a positive charge. In an example, the water-soluble group comprises a negative charge selected from sulfonic acid moiety, a carboxylic acid moiety, or a phosphate moiety or a positive charge selected from a quaternary ammonium moiety or other amine moieties. The branch terminator group may comprise any functional moiety, such as to functionalize the dendrimer. In some cases, the functional moiety may comprise a click chemistry group, such as DBCO, to attach the dendrimer to another object or surface that comprises the complementary click chemistry group, such as azide moieties. The cleavable linker portion may be any cleavable linker moiety described with respect to FIGS. 1-2. The stem linker portion may be any linker portion, multiples thereof, or combination thereof, described with respect to FIGS. 1-2.

The labelling reagent or labeled substrate may comprise any number of dendrimer branches, for example, at least about, at most about, and/or about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 or more dendrimer branches. The labelling reagent or labeled substrate may comprise any number of highest order dendrimer branches, for example, at least about, at most about, and/or about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 or more dendrimer branches. The labelling reagent or labeled substrate may comprise any number of optical moieties, for example, at least about, at most about, and/or about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 or more optical moieties.

FIG. 5B illustrates an example labelled substrate comprising a dUTP substrate with a cleavable linker “Y” (see FIG. 1), and a dendrimer core attached to each of (i) a stem linker portion comprising PEGₙ, (ii) two dendrimeric branches each comprising PEG₈, and (iii) an optical moiety comprising “Kam” (see FIG. 1). The stem linker portion may be attached to the cleavable linker portion. The optical moiety may be attached to the dendrimer core via an intermediate linker, an alkyl. Each dendrimer branch (e.g., PEG₈) is terminated by a water soluble group comprising a group of three sulfonic acid moieties. Each of the sulfonic acid moieties is negatively charged. The dendrimer core illustrated in FIG. 5B is a 3,5-dihydroxybenzamide group.

FIG. 5C illustrates an example labelled substrate comprising a dUTP substrate with a cleavable linker “Y” (see FIG. 1), and a dendrimer core attached to each of (i) a stem linker portion comprising PEGₙ, (ii) four dendrimeric branches each comprising PEG₈, and (iii) two optical moieties each comprising “Kam” (see FIG. 1). The stem linker portion may be attached to the cleavable linker portion. The optical moiety may be attached to the dendrimer core via an intermediate linker, an alkyl. Each dendrimer branch (e.g., PEG₈) is terminated by a water soluble group comprising a group of three sulfonic acid moieties. In some cases, each dendrimer branch (e.g., PEG₈) is terminated by a water soluble group comprising a group of one to three sulfonic acid moieties. Each of the sulfonic acid moieties is negatively charged. The dendrimer core illustrated in FIG. 5C is a 3,5-bis(aminomethyl)benzamide group.

The dendrimer core may refer to any atom or functional group, or any molecular segment containing therewith, that is attached to a first order dendrimer branch. For example, two or more first order dendrimer branches may be attached to the same atom or same functional group (e.g., different atoms of a benzene ring), or same molecular segment. While some examples of dendrimers described herein distinctly describe a linker (e.g., stem linker portion, cleavable linker portion) and a dendrimer core separately for ease of textual description, it will be appreciated that the linker and core need not be distinct from each other, and a linker or linker segment may comprise the dendrimer core and/or a dendrimer core may comprise a linker or linker segment.

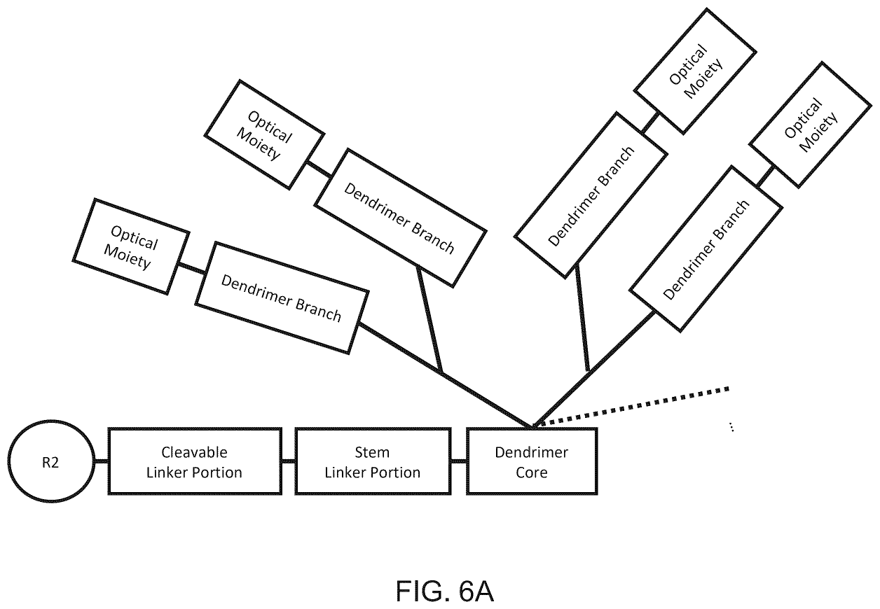

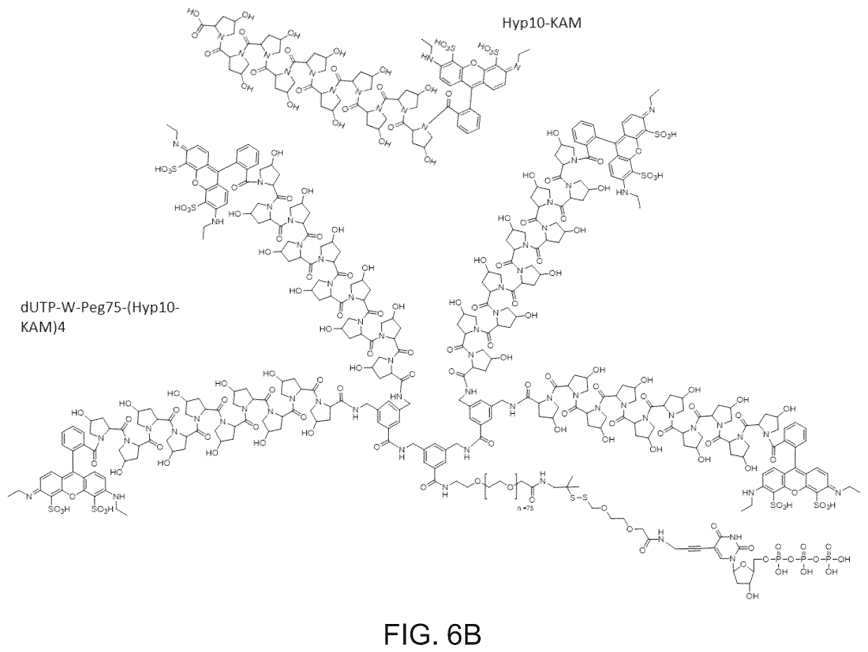

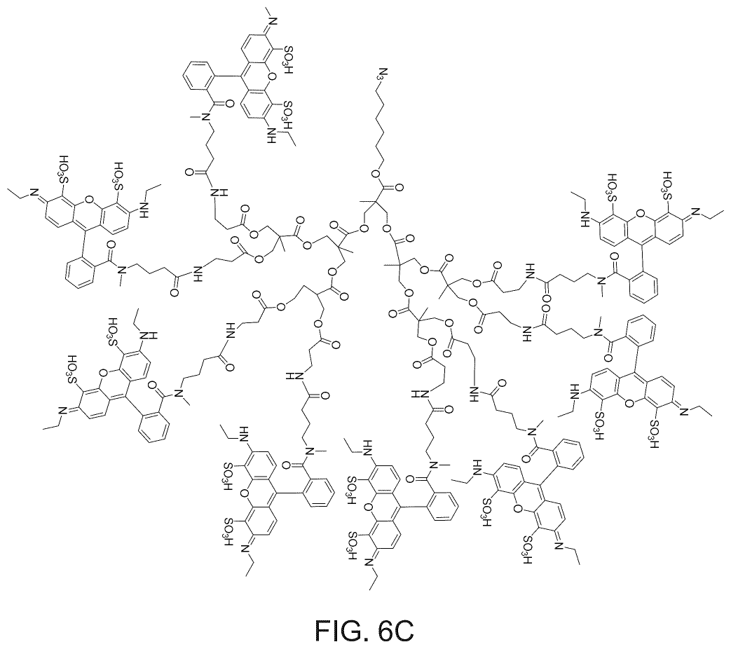

FIG. 6A illustrates another example dendrimeric labelled substrate comprising (i) a substrate “R2”, (ii) a linker comprising a cleavable linker portion, a stem linker portion, a dendrimer core, and dendrimeric branches, and (iii) an optical moiety. The dendrimer core may be covalently attached to one or more dendrimeric branches and the stem linker portion. A dendrimeric branch may be attached to the dendrimer core as a first order branch, second order branch, third order branch, or higher order branch. For example, second or higher order branches may be attached to the core via another lower order branch(es). In the example of FIG. 6A, each dendrimeric branch is a second order branch from the core (attached via a first order branch). Multiple dendrimeric branches attached to the dendrimer core may be of the same order or different order. A dendrimeric branch may comprise any linker segment of any length or any number of repeating units described elsewhere herein, such as a PEG segment or nonproteinogenic amino acid segment, such as hydroxyproline (Hypₙ), of any length. Alternatively or in addition, a dendrimeric branch may comprise any polymer or moiety of any length or any number of repeating units. For example, a dendrimeric branch may comprise a repeating polymer or moiety, having at least about, at most about, and/or about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more repeating units. In another example, a dendrimeric branch may comprise a hydroxyproline segment of any length, having at least about, at most about, and/or about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more repeating units. In some cases, a dendrimeric branch may itself comprise a dendrimer, comprising its own dendrimeric core and dendrimeric branches. Each dendrimeric branch may have the same or different lengths.

The optical moiety may be disposed at or near a distal end of a dendrimer branch relative to the dendrimer core. The optical moiety may be disposed at or near a distal end of a highest order dendrimer branch relative to the dendrimer core. Beneficially, where the dendrimer branch has a degree of rigidity, such as when comprising hydroxyproline, an optical moiety attached to one branch may be distanced from an additional optical moiety attached to another branch, even a directly neighboring branch, due to the dendrimeric branch structure. Sufficiently spaced apart optical moieties benefit from reduced quenching between such optical moieties, enabling detection of higher intensity (e.g., fluorescence intensity) with fewer number of dyes and stable optical resolution. The labelling reagent or labeled substrate may comprise multiple optical moieties, each optical moiety being disposed at or near an end of a respective dendrimer branch. In some cases, a dendrimer branch or each dendrimer branch may have a minimum length or minimum number of repeating units to ensure sufficient separation distance between neighboring optical moieties. The optical moieties may be the same or different optical moieties. The optical moieties may be the same or different charge.

The cleavable linker portion may be any cleavable linker moiety described with respect to FIGS. 1-2. The stem linker portion may be any linker portion, multiples thereof, or combination thereof, described with respect to FIGS. 1-2.

The labelling reagent or labeled substrate may comprise any number of dendrimer branches, for example, at least about, at most about, and/or about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 or more dendrimer branches. The labelling reagent or labeled substrate may comprise any number of highest order dendrimer branches, for example, at least about, at most about, and/or about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 or more dendrimer branches. The labelling reagent or labeled substrate may comprise any number of optical moieties, for example, at least about, at most about, and/or about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 or more optical moieties.

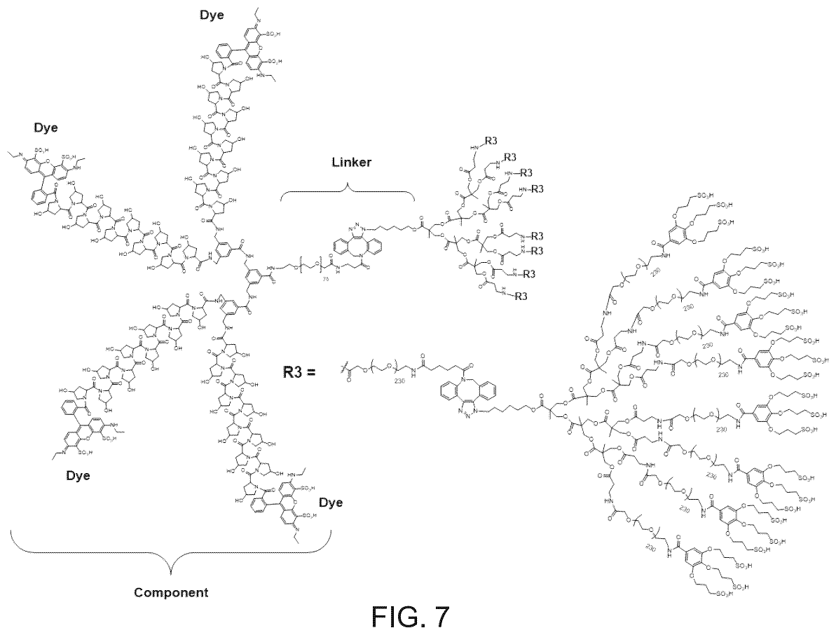

FIG. 6B illustrates in the bottom left an example labelled substrate comprising a dUTP substrate with the structure: dUTP-W-PEG₇₅-(Hyp10-Kam)₄ comprising a cleavable linker “W” (see FIGS. 1-2), and a dendrimer core attached to each of (i) a stem linker portion comprising PEG₇₅, (ii) two first order dendrimeric branches each further branching off into two second order dendrimeric branches for a total of four second order dendrimeric branches, each second order dendrimeric branch comprising a hyp10 segment, and (iii) an optical moiety comprising “Kam” (see FIG. 1) disposed at the end of each of the four highest order (second order) dendrimeric branches. The stem linker portion may be attached to the cleavable linker portion. The dendrimer core illustrated in FIG. 6B is a 3,5-dihydroxybenzoic acid group. The top right shows an example dendron with the structure: Hyp10-Kam, comprising a Kam dye attached to the end of a hyp10 segment. The illustrated dendron or molecules of other similar structures may be used to synthesize a dendrimeric branch. A dendrimeric branch may comprise the illustrated dendron or molecules of other similar structures. In some cases, a dendrimeric branch may itself comprise a dendrimer (e.g., including a dendrimer core and its own dendrimeric branches, see FIG. 7).

### Dendrimeric Carriers

A dendrimer described herein may carry, alternatively or in addition to optical moieties, any other agent, reagent, and/or component. A dendrimer may carry a single unit of a component. A dendrimer may carry multiple units or a colony of a component or a group of components. The component may be any amplification reagent and/or sequencing reagent described elsewhere herein, such as an oligonucleotide molecule, an adapter molecule, a barcode molecule, a UMI molecule, a primer molecule (e.g., sequencing and/or amplification), a template molecule, a probe molecule, an enzyme, a polymerase, a binder (e.g., via nucleic acid hybridization or other mechanism, e.g., electrostatic), a catalyst, a nucleotide reagent for incorporation and/or binding (e.g., dNTP, ddNTP, etc.), an oligonucleotide reagent for binding and/or ligation, etc. The component may be any other agent or reagent. In an example, a dendrimer comprises a plurality of optical moieties, which dendrimer is used to label a nucleotide substrate that is configured for incorporation during a sequencing reaction. In another example, a dendrimer comprises a plurality of primers that are configured for use in amplification of a library or template molecule. In another example, a dendrimer comprises a plurality of labeled nucleotide bases that are configured for incorporation during a sequencing reaction.

Provided herein is a method for delivering amplification reagents to a template nucleic acid, comprising: contacting the template nucleic acid with a solution comprising a plurality of dendrimers, wherein a dendrimer or each dendrimer of the plurality of dendrimers is attached to a plurality of amplification primers; and hybridizing an amplification primer of the plurality of amplification primers to the nucleic acid template molecule, or a derivative thereof, and extending the amplification primer. In some cases, the template nucleic acid may be immobilized to a substrate prior to the contacting with the dendrimers. In some cases, the method may further comprise generating an amplification colony at an individually addressable location of the substrate that the template nucleic acid is immobilized to. The substrate may comprise a plurality of individually addressable locations, with a plurality of template nucleic acid molecules each immobilized at a different location. Amplification, via contacting the plurality of template nucleic acid molecules with the solution comprising the plurality of dendrimers, may generate a plurality of amplification colonies at respective locations on the substrate. The method may further comprise sequencing the amplification colony. The sequencing may comprise extending a sequencing primer (or growing strand) hybridized to the template molecule by providing nucleotide reagents, washing away unincorporated nucleotides from the reaction space, and detecting signals indicative of incorporation, or lack thereof. Alternatively, any other sequencing method may be used.

In some cases, a dendrimer may carry a spacer and/or may itself function as a spacer. For example, a dendrimer may be attached to a template molecule at its core and the dendrimer branches may carry a spacer (e.g., PEG segment, hyp segment, etc.) and/or may themselves act as a spacer, where the template molecule is configured to be sequenced by hybridizing a sequencing primer, and the dendrimer spaces out the template molecule from a neighboring template molecule (optionally also attached to a dendrimer) via the spacer. Thus, the dendrimers may enable physical, spatial resolution between neighboring template molecules. In some cases, the dendrimers may not be attached to template molecules, but instead comprise or be attached to a binding agent (e.g., an oligonucleotide molecule, a functional group, such as a click chemistry reagent, etc.), and comprise or be a spacer. A plurality of such dendrimers may space each other out, and be immobilized or spatially restricted, and thereafter a plurality of components capable of binding to the binding agent may be provided to the plurality of dendrimers, thus binding the components to the dendrimers and spacing out the components via the dendrimers.

Provided herein is a method for physically, spatially separating nucleic acid template molecules on a substrate, comprising: loading and immobilizing a plurality of dendrimers as a layer on the substrate, wherein a first dendrimer of the plurality of dendrimers is attached to a nucleic acid template molecule, and wherein the first dendrimer comprises a plurality of dendrimeric branches that spaces the template molecule from an additional template molecule attached to a second dendrimer immobilized adjacent to the dendrimer. The method may further comprise sequencing the template molecule. The sequencing may comprise extending a sequencing primer (or growing strand) hybridized to the template molecule by providing nucleotide reagents, washing away unincorporated nucleotides from the reaction space, and detecting signals indicative of incorporation, or lack thereof. Alternatively, any other sequencing method may be used. Provided herein is a method for physically, spatially separating nucleic acid template molecules on a substrate, comprising: loading and immobilizing a plurality of dendrimers as a layer on the substrate, wherein a first dendrimer of the plurality of dendrimers comprises or is attached to a binding agent, and wherein the first dendrimer comprises a plurality of dendrimeric branches that spaces the binding agent from an additional binding agent of or attached to a second dendrimer immobilized adjacent to the dendrimer; and contacting the plurality of dendrimers immobilized to the substrate with a plurality of template nucleic acid molecules, thereby binding template nucleic acid molecules of the plurality of template nucleic acid molecules to a plurality of binding agents. The method may further comprise sequencing the template molecule. The sequencing may comprise extending a sequencing primer (or growing strand) hybridized to the template molecule by providing nucleotide reagents, washing away unincorporated nucleotides from the reaction space, and detecting signals indicative of incorporation, or lack thereof. Alternatively, any other sequencing method may be used.

A dendrimer may have any dimension. In some cases, the maximum dimension of a dendrimer, such as a maximum diameter, may be at least about, at most about, and/or about 0.1 nm, 0.2 nm, 0.3 nm, 0.4 nm, 0.5 nm, 0.6 nm, 0.7 nm, 0.8 nm, 0.9 nm, 1.0 nm, 2 nm, 3 nm, 4 nm, 5 nm, 6 nm, 7 nm, 8 nm, 9 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm or more.

FIG. 7 illustrates an example multi-layer dendrimer structure. A multi-layer dendrimer may be used as a dendrimeric carrier of a component (e.g., optical moiety, reagents, etc.) as described elsewhere herein. Alternatively or in addition, a multi-layer dendrimer may be used as a spacer, as described elsewhere herein. In the example structure, a dendrimer core (2,2 bis(hydroxymethyl) propionic acid (bis-MPA)) is attached to (1) a component (a multi-labeled dendrimer) via a linker (comprising PEG₇₅) and (2) two first order dendrimeric branches, where each first order dendrimeric branch branches off to two second order dendrimeric branches for a total of four second order dendrimeric branches, where each second order dendrimeric branch branches off to two third order dendrimeric branches for a total of eight third order dendrimeric branches, and where each third order dendrimeric branch comprises a dendrimer “R3”. Dendrimer “R3”, structure shown in the bottom right of FIG. 7, comprises a dendrimer core (bis-MPA) attached to two first order dendrimeric branches, which branch to four second order dendrimeric branches, which branch to eight third order dendrimeric branches, where each third dendrimer branch comprises PEG₂₃₀ and is terminated by a water soluble group comprising a group of three sulfonic acid moieties. In some cases, the charged group (e.g., sulfonic acid moieties) terminating the highest order branches of the multi-layer dendrimer may be configured to bond to a substrate to immobilize the multi-layer dendrimer, and thus the component (e.g., multi-labeled dendrimer), to the substrate. Alternatively or in addition, the branch terminator group may comprise any functional moiety, such as to functionalize the dendrimer. In some cases, the functional moiety may comprise a click chemistry group, such as DBCO, to attach the dendrimer to another object or surface that comprises the complementary click chemistry group, such as azide moieties. It will be appreciated that a multi-layer dendrimer may carry a component that is not a multi-labeled dendrimer or other optical moiety. For example, a multi-layer dendrimer may be attached to a template molecule or other reagent. The illustrated molecule may thus comprise 2 first order branches, 4 second order branches, 8 third order branches, 16 fourth order branches, 32 fifth order branches, and 64 sixth order (highest order) branches in the dendrimer to the right.

A dendrimer may comprise layers of branches to any order, for example about, at least about, and/or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more orders.

### Labeled Proteins

A labelling reagent may comprise a linker comprising a protein and an optical moiety. For example, the labelling reagent may comprise a labelled protein, which is labeled with an optical moiety. A labeled substrate may comprise a substrate, such as a nucleotide, coupled to the labelling reagent.

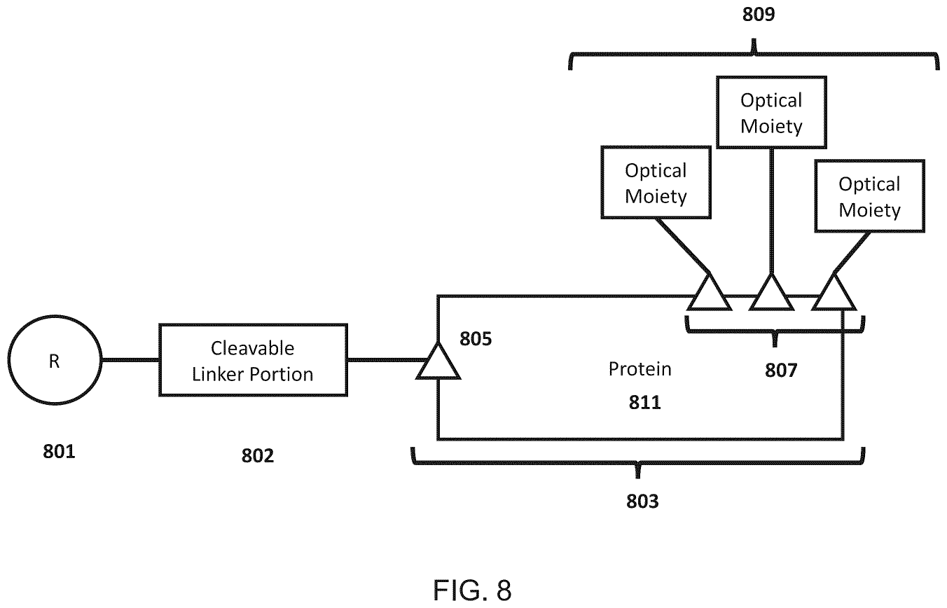

FIG. 8 illustrates a labeled substrate comprising a substrate 801 coupled to a linker comprising (i) a cleavable linker portion 802, and (ii) a labeled protein 803. The labeled protein 803 may comprise (i) a protein 811, (ii) a substrate attachment site 805, which attaches to the substrate 801 via the cleavable linker portion 802, and (iii) an optical moiety attachment site 807, which attaches to an optical moiety 809. The cleavable linker portion may be any cleavable linker moiety described with respect to FIGS. 1-2. The substrate attachment site 805 may be native to or engineered within the protein 811. The substrate attachment site 805 may be external to and coupled to the protein 811. The optical moiety attachment site 807 may be native to or engineered within the protein 811. The optical moiety attachment site 807 may be external to and coupled to the protein 811.

The labeled protein 803 may comprise one or more substrate attachment sites (e.g., 805), for example at least about, at most about, and/or about 1, 2, 3, 4, 5, 6, 7, 8, 9, 10 or more sites. Where there are multiple substrate attachment sites, none, some, or all of the sites may be native to or engineered within the protein 811, and/or none, some, or all of the sites may be external to and coupled to the protein 811. The labeled protein 803 may comprise one or more optical moiety attachment sites (e.g., 807), for example at least about, at most about, and/or about 1, 2, 3, 4, 5, 6, 7, 8, 9, 10 or more sites. Where there are multiple optical moiety attachment sites, none, some, or all of the sites may be native to or engineered within the protein 811, and/or none, some, or all of the sites may be external to and coupled to the protein 811.

The labeled protein 803 may be attached to one or more optical moieties (e.g., 809), for example at least about, at most about, and/or about 1, 2, 3, 4, 5, 6, 7, 8, 9, 10 or more moieties. In some cases, the labeled protein may have the same number of optical moieties as optical moiety attachment sites. In some cases, the labeled protein may have a larger number of optical moieties as optical moiety attachment sites. In some cases, the labeled protein may have a smaller number of optical moieties as optical moiety attachment sites. For example, a single optical moiety may be attached to a single optical moiety attachment site, a single optical moiety may be attached to multiple optical moiety attachment sites, multiple optical moieties may be attached to a single optical moiety attachment site, each optical moiety attachment site may be attached to at least one optical moiety, and/or some optical moiety attachment sites may be unattached to any optical moiety. In some cases, the multiple optical moieties may comprise dendrimeric optical moieties as described herein.

The protein 811 may natively comprise the substrate attachment site and/or the optical moiety attachment site. Alternatively or in addition, a substrate attachment site and/or an optical moiety attachment site may be engineered. An attachment site may be engineered within the protein or be external to and coupled to the protein 811. In an example, the substrate attachment site is a serine, glycine, or cysteine residue. In an example, the substrate attachment site is an N-terminal serine, glycine, or cysteine residue. In an example, the optical moiety attachment site is a cysteine or lysine residue. In an example, the optical moiety attachment site is a C-terminal cysteine or lysine residue. Examples of optical moiety or substrate attachment sites include glycine, methionine, arginine, cysteine, tryptophan, tyrosine, lysine, serine and histidine amino acids. Of course, in each case, the attachment entities (e.g., cleavable linker portion 802, optical moiety 809) have complementary reactive groups. For example, the amines of lysines and thiols of cysteines may be used to attach to the attachment entities. In some cases, aromatic residues (e.g., tryptophan, tyrosine, histidine) or sulfur residues (e.g., methionine) may be added in the protein as targets for reactive oxygen species. Where there are multiple substrate attachment sites, the sites may be the same or different type (e.g., residues) of sites. Where there are multiple optical moiety attachment sites, the sites may be the same or different type (e.g., residues) of sites. A substrate attachment site and an optical moiety attachment site may be the same type (e.g., residue) or different types (e.g., residues) of sites. In some cases, the substrate attachment site(s) may be disposed at or near the N-terminal and the optical moiety attachment site(s) may be disposed at or near the C-terminal. Alternatively, the substrate attachment site(s) may be disposed at or near the C-terminal and the optical moiety attachment site(s) may be disposed at or near the N-terminal. Being disposed near a terminal may refer to a location that is e.g., within 20, 19, 18, 17, 16, 15, 14, 13, 12, 11, 10, 9, 8, 7, 6, 5, 4, 3, 2, 1, or 0 residues from the terminal residue. Being disposed near a terminal may refer to a location that is e.g., within 20, 19, 18, 17, 16, 15, 14, 13, 12, 11, 10, 9, 8, 7, 6, 5, 4, 3, 2, 1, or 0 Angstroms (Å) from the terminal residue. Beneficially, the native or engineered structure of a protein may provide a rigid or semi-rigid separation between the substrate and the optical moiety. Alternatively or in addition, a substrate attachment site and/or optical moiety attachment site may be a native internal residue of the protein and/or disposed at the globular portion of the protein.

The protein 811 and/or the labeled protein 803 may have at least about, at most about, and/or about 10, 20, 30, 40, 50, 60, 70, 80, 90, 100, 150, 200, 250, 300, 350, 400, 450, 500 or more amino acids. Alternatively or in addition, the protein 811 and/or the labeled protein 803 may have a molecular mass of at least about, at most about, or about 1 k, 2 k, 3 k, 4 k, 5 k, 6 k, 7 k, 8 k, 9 k, 10 k, 11 k, 12 k, 13 k, 14 k, 15 k, 16 k, 17 k, 18 k, 19 k, 20 k, 25 k, 30 k, 35 k, 40 k, 45 k, 50 k or more Daltons (Da). In some cases, the protein 811 and/or the labeled protein 803 may have at most about 10, 20, 30, 40, 50, 60, 70, 80, 90, 100, 150, 200, 250, 300, 350, 400, 450, 500 or less amino acids. In some cases, the protein 811 and/or the labeled protein 803 may have a molecular mass of at most about 1 k, 2 k, 3 k, 4 k, 5 k, 6 k, 7 k, 8 k, 9 k, 10 k, 11 k, 12 k, 13 k, 14 k, 15 k, 16 k, 17 k, 18 k, 19 k, 20 k, 25 k, 30 k, 35 k, 40 k, 45 k, 50 k or less Daltons (Da). In some cases, the protein may have at least one dimension that is at least about 25 Angstroms (Å), 50 Å, 75 Å, 100 Å, 125 Å, 150 Å, or 200 Å in length. In some cases, the distance between the N-terminus to the C-terminus of the protein (in folded protein form) may be at least about 25 Angstroms (Å), 50 Å, 75 Å, 100 Å, 125 Å, 150 Å, or 200 Å in length. In some cases, the minimum distance between a substrate attachment site and an optical moiety attachment site may be at least about 25 Angstroms (Å), 50 Å, 75 Å, 100 Å, 125 Å, 150 Å, or 200 Å in length. In some cases, the protein 811 and/or the labeled protein 803 may have a maximum dimension of at most about 1000 Å, 900 Å, 800 Å, 700 Å, 600 Å, 500 Å, 450 Å, 400 Å, 350 Å, 300 Å, 250 Å, 200 Å, 150 Å, 100 Å or less.

In some cases, the protein in the labelling reagent may have any number of optical moiety attachment sites, for example, at least about, at most about, and/or about 1, 2, 3, 4, 5, 6, 7, 8, 9, 10 or more optical moiety attachment sites. In one example, cysteine residues may be used as optical moiety attachment sites. In some cases, the protein in the labelling reagent may have any number of engineered or predetermined cysteine residues for use as optical moiety attachment sites, for example, at least about, at most about, and/or about 1, 2, 3, 4, 5, 6, 7, 8, 9, 10 or more cysteine residues. In some cases, the protein in the labelling reagent may have any total number of cysteine residues, including native and engineered, for use as optical moiety attachment sites, for example, at least about, at most about, and/or about 1, 2, 3, 4, 5, 6, 7, 8, 9, 10 or more cysteine residues. In some cases, the protein lacks any native cysteine residues (or other residues used for the purposes of substrate attachment). Attachment residues may be created by mutating selected residues in the example proteins. In some cases, the protein in the labelling reagent may be engineered to remove or modify native cysteines in the protein amino acid sequence to achieve a desired number of optical moiety attachment sites. The removal and/or addition of cysteines (or other optical moiety attachment sites) may be performed to locate attachment sites at desired locations (e.g., on the outer edge(s) of folded proteins, separate from substrate attachment site(s), and/or distant from other optical moiety attachment site(s), etc.). By way of example, a substrate attachment site may be located at the N-terminal of the protein and the optical moiety attachment site(s) may be located at the C-terminal of the protein. Similarly, where cysteine, glycine, methionine, arginine, tryptophan, tyrosine, lysine, serine and/or histidine residue(s) are used as attachment site(s), for optical moieties or substrates, such residues may be native or engineered, and/or added, mutated, modified, and/or removed to achieve any predetermined number (e.g., 1, 2, 3, 4, 5, 6, 7, 8, 9, 10 or more) or predetermined location(s) in the labelled protein.

In some cases, multiple optical moiety attachment sites may be separated by rigid or semi-rigid linkers. For example, multiple optical moiety attachment sites may be separated by any of the linker portions, multiples thereof, and/or combinations thereof described with respect to FIGS. 1-2. In some cases, multiple optical moiety attachment sites may be separated by polyproline or other helices. In some cases, multiple optical moiety attachment sites may be separated by EAAAK linker(s).

Some example proteins include small ubiquitin-like modifier (SUMO) proteins, maltose-binding proteins (MBP), thioredoxin, streptavidin, etc. In some cases, the protein is a globular protein. In some cases, the protein is monomeric. Alternatively, the protein is multimeric (e.g., a dimer, tetramer, etc.). In some such cases, where the protein is multimeric, each monomer comprises a same number of optical moiety attachment sites (e.g., 1, 2, 3, 4, 5, etc.).

In the case of labelling streptavidin, in some cases, the streptavidin may be modified to precisely label sub-units with optical moieties, for example modifications with cysteines.

A labelling reagent or labeled substrate comprising a labeled protein may comprise any of the linker portions, multiples thereof, and/or combinations thereof described with respect to FIGS. 1-2, such as between the cleavable linker portion and the labeled protein or between different optical moiety attachment sites.

FRET with Multiple Optical Moieties

Provided herein are fluorescence resonance energy transfer (FRET)-based detection schemes that may be used with the substrates and labelling reagents described herein. Any sequencing method described herein may comprise a FRET-based detection method. In some cases, a substrate (e.g., nucleotide) may be labelled with multiple optical moieties of the same type or different type (e.g., labelled with different dyes). The labelling reagents used in FRET-based detection methods may comprise, for example, dendrimeric labels and protein labels as described elsewhere herein.

In a sequencing or other analytical method that comprises FRET-based detection, different substrates such as the different canonical base types of nucleotides (e.g., A, G, C, T, U, etc.), may be encoded via any one or combination of multiple dyes. In some cases, a set of different substrates may be encoded via a single type of dye, such as via adjusting the number and/or intensity of the dye for each type of substrate. Such encoding scheme may be decoded via greyscale (or single frequency) calibration and detection. Alternatively or in addition, a set of different substrates may be encoded via a combination of at least and/or at most 2, 3, 4, 5, 6, 7, 8, 9, 10 or more different types of dyes. In some cases, four nucleotide types may be encoded via at least and/or at most 1, 2, 3, 4, 5, 6, 7, 8, 9, 10 or more different types of dyes. In some cases, a FRET-based detection method may comprise a single excitation channel or multiple excitation channels. In some cases, a FRET-based detection method may comprise at least and/or at most 1, 2, 3, 4, 5, 6, 7, 8, 9, 10 or more excitation channels. In some cases, a FRET-based detection method may comprise a single excitation channel or multiple emission channels. In some cases, a FRET-based detection method may comprise at least and/or at most 1, 2, 3, 4, 5, 6, 7, 8, 9, 10 or more emission channels. In some cases, a FRET-based detection method may comprise the same number of emission channels as excitation channels. In some cases, a FRET-based detection method may comprise a different number of emission channels as excitation channels. In some cases, a FRET-based detection may comprise the same number of emission channels and/or excitation channels as the number of dyes encoding the substrates. In some cases, a FRET-based detection may comprise a different number of emission channels and/or excitation channels as the number of dyes encoding the substrates.

Example 6 illustrates example excitation and emission channel detection schemes for substrates that may be labelled with streptavidin that is labelled with different combinations of AZ555 and AZ647 dyes.

### Protecting Groups for Fluorescence Stability

Fluorescent dyes may exhibit intermittent fluorescence, also referred to herein as “blinking”, and photo-degradation, or “photobleaching” or “photodamage”. Such behavior may negatively impact detection of the fluorophore, greatly decreasing the reliability of an optical signal detected or lack thereof, and thus greatly decreasing accuracy of downstream sequencing analysis. Provided are systems and methods to address at least the abovementioned problems as well as quenching.

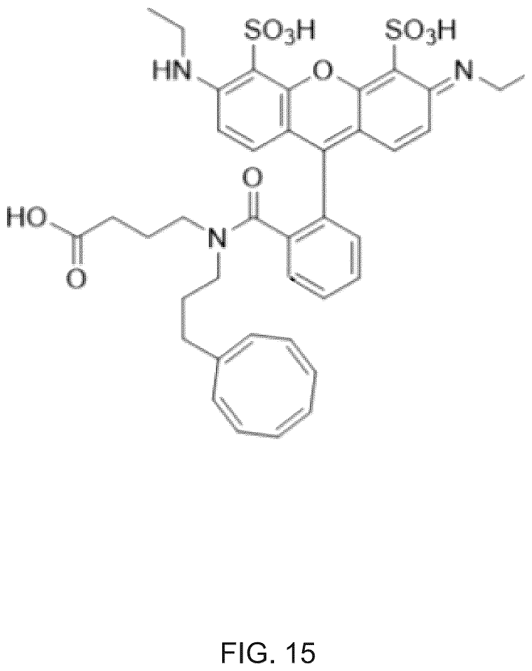

A protecting group may be attached to an amino acid to generate a protecting reagent, which protecting reagent can be modularly attached between a linker and an optical moiety (e.g., fluorescent dye) for a labelled substrate. The protecting group in the protecting reagent may significantly reduce the lifetime of a fluorophore's triplet state, thereby improving the blinking problem. A protecting group may comprise cyclooctatetraene (COT). The mechanism of triplet lifetime reduction of a COT protecting group on the Cy5 fluorophore is described in Qinsi Zheng et al., On the Mechanisms of Cyanine Fluorophore Photostabilization, J. Phys. Chem. Lett. 3(16), Aug. 16, 2012, which is entirely incorporated by reference herein for all purposes. Beneficially, the amino acid attached to the protecting group in the protecting reagent greatly increases the modularity and ease of attachment of the protecting reagent to various linker and label components described elsewhere herein. For example, the protecting reagent may be easily attached with other components described with respect to FIGS. 1-2. FIG. 15 illustrates an example protected fluorophore comprising a COT protecting reagent covalently attached to a KAM dye. The protected fluorophore can now modularly be attached to an amino acid linker (e.g., Hypₙ) and/or cleavable linker which is attached to a substrate (e.g., dUTP).

### Terminators

It will be appreciated that any of the nucleotide substrates described herein, labeled or unlabeled, may or may not comprise a terminator. The terminator may be a reversible terminator, irreversible terminator, or any other terminator described elsewhere herein, such as a virtual terminator. Terminated or non-terminated nucleotides, whether labelled or unlabeled, may be used as sequencing reagents.

### Kits & Compositions

Provided herein are kits and compositions comprising sequencing reagents described herein. A kit and/or composition may comprise any of the labelling reagents or labelled substrates described herein, such as labelling reagents or labelled substrates comprising PEG or modified PEG, and/or labelling reagents or labelled substrates comprising labeled proteins. In some cases, where the substrate is a nucleotide, a kit or composition may comprise 1, 2, 3, 4, or 5 (A, T, G, C, U) canonical base types. In some cases, a combination of different kits or compositions of single base types may be provided. A kit or composition may comprise any component, such as a linker moiety or component, PEG, protein, an optical moiety (e.g., fluorescent dye), or the like, of the labelling reagents or labelled substrates. A kit or composition may comprise any reagent useful for sequencing or amplification, such as labeled nucleotides, unlabeled nucleotides, buffers, polymerases, enzymes, catalysts, beads, etc. A kit or composition, or any component thereof, may exist in solution. A kit or composition, or any component thereof, may exist at least partially in solid phase. A kit or composition, or any component thereof, may be immobilized to a surface, such as of a bead or substrate, of any surface area, for example, about, at least about, and/or at most about 0.01, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 104, 105, 106 square millimeters (mm²) or more.

### Chemical Scars

Cleavage of a cleavable group may leave a scar group associated with substrate. The cleavable group can be, for example, an azidomethyl group capable of being cleaved by an agent such as tris(2-carboxyethyl)phosphine (TCEP), dithiothreitol (DTT), or tetrahydropyranyl (THP) to leave a hydroxyl scar group. The cleavable group can be, for example, a disulfide bond capable of being cleaved by an agent such as TCEP, DTT or THP to leave a thiol scar group. The cleavable group can be, for example, a hydrocarbyldithiomethyl group capable of being cleaved by an agent such as TCEP, DTT or THP to leave a hydroxyl scar group. The cleavable group may comprise a photocleavable moiety. For example, the cleavable group can be, for example, a 2-nitrobenzyloxy group capable of being cleaved by ultraviolet (UV) light to leave a hydroxyl scar group. A linker or a labeled reagent comprising the linker may be stable in the absence of an agent, light (e.g., ultraviolet light), or condition (e.g., a particular pH range) capable of cleaving a cleavable linker. For example, a linker comprising a cleavable disulfide group may be stable in the absence of a reducing agent.

A residual portion of a linker remaining on the substrate following cleavage of the cleavable group may be referred to as a ‘scar’ or as a cleaved linker. In an example, prior to labeling, a substrate may be functionalized to include a functional handle that is subsequently used to couple the substrate to a linker. Following cleavage and a post-cleavage reaction (e.g., an immolation reaction), such a functional handle may be part of a scar or a cleaved linker. A scar of a biomolecule (e.g., nucleotide) may comprise a portion of the biomolecule not typically associated with a canonical biomolecule of the same type (e.g., A, T, G, C, U nucleotide).

In some cases, a scar may alter a property of a substrate. For example, a scarred (i.e., scar-containing) nucleotide within a nucleic acid may inhibit further nucleotide incorporations into the nucleic acid. The scarred nucleotide may inhibit nucleotide incorporations at an immediately adjacent open position or may inhibit multiple subsequent nucleotide additions. In some cases, a scar may affect an optical property of a substrate. For example, a scar may quench fluorescence activity. In some cases, a scar may be reactive toward another species in a system, which may alter the performance of a system. For example, a nucleotide-bound scar may comprise a reactivity toward lysines, and thereby inhibit polymerase activity in a system. Thus, in some cases, results and performance of downstream operations (e.g., sequencing) can be enhanced by optimizing a scar's structure and properties. Chemical scars and various methods for addressing them are described in further detail in International Patent Pub. No. WO2022/212408A1, which is entirely incorporated herein by reference.

A scar may be stable upon cleavage. A scar may also be reactive. The scar's reactivity may be an intramolecular reactivity. In such cases, a scar may undergo a post-cleavage reaction to form a structure distinct from the initial scar formed upon cleavage. Such a post-cleavage reaction may be referred to as “immolation,” and scars which have undergone immolation may be referred to as “immolated scars.” In some cases, a scar may disappear altogether post-immolation. A linker may spontaneously immolate (i.e., undergo immolation) upon cleavage, or may form a first scar that is stable until it is contacted with a reagent or a specific condition (e.g., a specific pH range). Immolation may change a physical or chemical property of the scar group, and further may diminish its size. An immolated scar may comprise different properties than the post-cleavage scar from which it formed, which may make the immolated scar more favorable for a particular assay. In some cases, an immolated scar may inhibit an enzymatic activity (e.g., polymerase activity) less than the post-cleavage scar from which it formed. For example, using BST type polymerases for incorporations, thiol and propargyl alcohol scars (which can form directly from linker cleavage) can inhibit polymerization more than propargyl amine and primary aliphatic amine scars (which may be formed through scar immolation). In some cases, a less acidic scar (e.g., a scar comprising a higher pH) may inhibit an enzymatic activity less than a more acidic scar. In some cases, a smaller (e.g., lower mass, volume, or length) scar may inhibit an enzymatic activity less than a more acidic scar.

A strategy for mitigating an adverse effect of a scar is scar immolation. A scar may be configured to undergo a reaction subsequent to cleavage (e.g., an immolation reaction), which may alter a chemical or physical property of the scar. The immolation reaction may be initiated or accelerated by a reagent (e.g., a catalyst or reagent), light, or a condition (e.g., a pH range). The immolation reaction may be spontaneous. The immolation reaction may diminish the size of the scar. For example, an immolation reaction of a thiol-containing scar may result in the loss of the thiol moiety as a thiirane or thietane. As such, an immolation reaction may diminish the steric bulk of a scar. An immolation reaction may alter a chemical or physical property of a scar. For example, a thiol-containing scar may form a more polar and less acidic propargyl amine scar upon immolation. In some cases, a scar may be a thiol scar. In some cases, a scar may undergo an immolation scar to yield an immolated scar which comprises a primary amine or a primary hydroxyl moiety (e.g., comprising propargyl alcohol).

An alternative or additional strategy for mitigating an adverse (e.g., an inhibitory or mispair-inducing) effect of a scar is scar-capping. A physical or chemical property of a scar may be altered by coupling the scar to a capping reagent. The altered property may be favorable (e.g., relative to the uncapped, scarred substrate) for nucleic acid polymerization. For example, the altered physical or chemical property may diminish the inhibitory effect of a scar. The altered physical or chemical property may diminish the rate of nucleotide misincorporation into a growing nucleic acid molecule comprising the capped scar. Accordingly, a sequencing method may comprise, contacting a nucleic acid molecule complex (e.g., sequencing primer-template nucleic acid complex which has incorporated a labeled substrate) with a capping reagent. A capping reagent may be selective for a scar, and therefore may be added with a labeled nucleotide substrate, with a cleavage reagent, or subsequent to a cleavage reagent. A nucleic acid polymerization method may comprise a capping reagent addition prior to or following a labeled nucleotide incorporation. In some cases, a scar comprises a thiol scar. The capping reagent may comprise a disulfide configured to react with the thiol scar.

The capping reagent may be added with a labeled nucleotide, unlabeled nucleotide, with a cleavage reagent, subsequent to a cleavage reagent, or subsequent to a reagent, light-input, energy-input, or change in condition for a scar immolation reaction. The capping reagent may be added subsequent to a labeled nucleotide. The capping reagent may be added with an unlabeled nucleotide. For example, a method may comprise first contacting a nucleic acid with a labeled nucleotide, and then subsequently contacting the nucleic acid with a capping reagent and an unlabeled nucleotide of the same canonical type as the labeled nucleotide. Such a method may increase the likelihood of complete extension across homopolymeric regions of a template nucleic acid. The capping reagent may remain stably bound to the scarred nucleotide through subsequent nucleotide additions and cleavage steps.

The capping reagent may covalently (e.g., for a bond with) or non-covalently couple to the scar group. A capping reagent may covalently couple to a nucleophilic moiety on a scar, such as a hydroxyl or thiol. A capping reagent may reversibly or irreversibly couple to a scar. Examples of reversibly-binding capping reagents (“reversible capping reagents) include

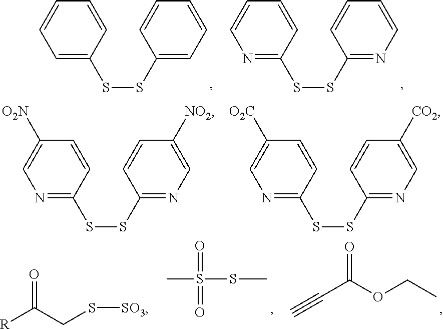

and optionally substituted (e.g., alkylated, halogenated, or carboxylated) variants thereof. It will be appreciated that examples of capping reagents include various isomers of the above, such as the 2-isomers and 4-isomers (e.g., pyridyldithio isomers), and their optionally substituted variants. Some examples of 4-isomers include:

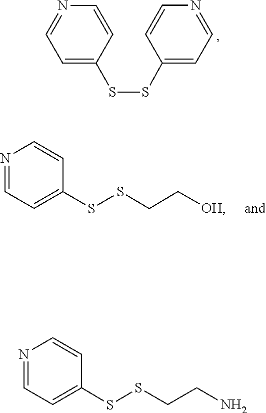 [SMILES: c1cc(SSc2ccncc2)ccn1 ; OCCSSc1ccncc1 ; "and" ; NCCSSc1ccncc1]

or (4-(4-pyridyldithio)pyridine), (2-(4-pyridyldithio) ethanol), and 2-(4-pyridyldithio)ethylamine.

A reversible thiol capping reagent may comprise a disulfide, a thiosulfate, or an alkyne, and may cap a thiol scar through for example a thiol-disulfide exchange or a thiol-yne reaction. Reversible capping of a thiol scar may convert the thiol into a disulfide. The disulfide may subsequently be cleaved by a reducing agent, such as THP. In some cases, a single reagent may cleave a cleavable linker and remove a reversible capping reagent. For example, a reducing reagent such as THP may remove a thiolate (e.g., a pyridine thiolate derived from a dipyridyldisulfide capping reagent or a benzenethiolate derived from a dibenzyldisulfide capping reagent). A capping reagent or a portion thereof (e.g., a methyl or acetyl group) may irreversibly couple to a scar. In some cases, irreversible coupling denotes formation of a stable bond in the conditions of and upon contact with the reagents for a particular assay. For example, a hydroxyl scar methylating reagent may be an irreversible capping reagent in a nucleic acid polymerization assay if none of the conditions or reagents of the assay are configured to remove a methyl group from a methoxide moiety. An irreversible thiol capping reagent may comprise an iodoacetyl or pyrrole dione moiety. Examples of irreversible thiol capping reagents include

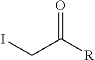

(wherein R may comprise O, S, optionally substituted alkyl, optionally substituted alkenyl, optionally substituted alkynyl, optionally substituted amine, optionally substituted alkoxide, cycloalkyl, optionally substituted heterocycloalkyl, optionally substituted aryl, and optionally substituted heteroaryl),

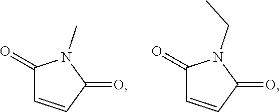

and optionally substituted (e.g., alkylated, halogenated, or carboxylated) variants thereof. An irreversible thiol capping reagent may comprise a substitutable halogen (e.g., iodide in iodoacetamide) or an electrophilic olefin (e.g., the double bonded carbons of a pyrrole dione), and may form a carbon-sulfur bond between the thiol scar and the capping reagent or a portion thereof. Some example capping reagents include, but are not limited to, ethyl propiolate (EP), iodoacetamide (IAc), methyl methanethiosulfonate (MMTS), and dipyridyl disulfide (DPDS), 4-4′-dipyridyl disulfide, 2,2′-dithiobis(5-nitropyridine), 4-4′-dipyridyl disulfide, 2,2′-dithiobis(5-nitropyridine), 6,6′-dithiodinicotinic acid, and pyridyl ethyl amine disulfide (PEAD).

### Hydroxyl Scars

Several cleavable linker moieties described in the present disclosure, upon cleavage, leave a hydroxyl scar which comprises an —OH moiety. Beneficially, the hydroxyl moiety may be relatively less reactive, for example compared to a thiol scar (comprising a —SH moiety). For the purposes of sequencing reactions where for example, the substrate comprises a dNTP, the hydroxyl scar may be significantly less inhibitive than the thiol scar.

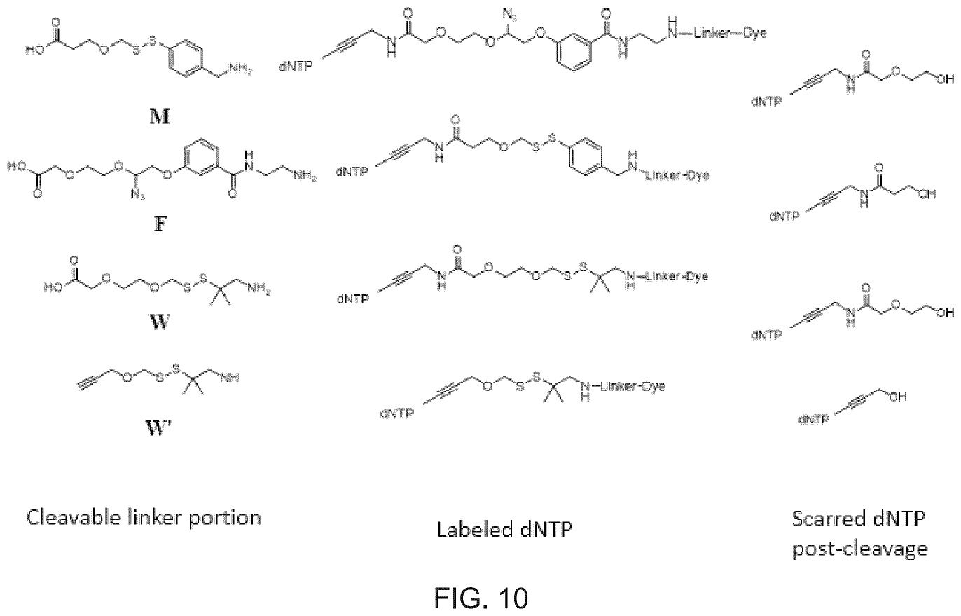

FIG. 10 illustrates the hydroxyl scar on a dNTP substrate upon cleavage of a linker comprising cleavable linker components derived from the M, F, W, or W′ cleavable linker moieties (see FIGS. 1-2 for the cleavable linker moieties). It will be appreciated that the substrate may be any substrate described herein, alternatively or in addition to the dNTP illustrated in FIG. 10.

### Sequencing Using Labeled Substrates

The labeled substrates of the present disclosure may be used to sequence a template nucleic acid. For example, the labeled substrates comprise labeled nucleotides. The template nucleic acid may be sequenced while attached to a support (e.g., bead). Alternatively, the template nucleic acid may be free of the support when sequenced and/or analyzed. The template nucleic acid may be sequenced while immobilized to a substrate, such as via a support or otherwise. Any sequencing method may be used, for example pyrosequencing, single molecule sequencing, sequencing by synthesis (SBS), sequencing by ligation, sequencing by binding, non-terminated sequencing, flow-based sequencing, terminated sequencing, etc. Sequencing may be performed on an amplified molecule (e.g., concatemers), amplified colony (e.g., amplicons), and/or on single molecules.

Sequencing may comprise extending a sequencing primer (or growing strand) hybridized to a template nucleic acid by providing labeled nucleotide reagents, washing away unincorporated nucleotides from the reaction space, and detecting one or more signals from the labeled nucleotide reagents which are indicative of an incorporation event or lack thereof. After detection, the labels may be cleaved and the whole process may be repeated any number of times to determine sequence information of the template nucleic acid. One or more intermediary flows may be provided intra- or inter-repeat, such as washing flows, label cleaving flows, terminator cleaving flows, reaction-completing flows (e.g., double tap flow, triple tap flow, etc.), labeled flows (or bright flows), unlabeled flows (or dark flows), phasing flows, chemical scar capping flows, etc. A nucleotide mixture that is provided during any one flow may comprise only labeled nucleotides, only unlabeled nucleotides, or a mixture of labeled and unlabeled nucleotides. The mixture of labeled and unlabeled nucleotides may be of any fraction of labeled nucleotides, such as at least or at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A nucleotide mixture that is provided during any one flow may comprise only non-terminated nucleotides, only terminated nucleotides, or a mixture of terminated and non-terminated nucleotides. When using only non-terminated nucleotides, terminator cleaving flows may be omitted from the sequencing process. When using terminated nucleotides, to proceed with the next step of extension, prior to, during, or subsequent to detection, a terminator cleaving flow may be provided to cleave blocking moieties. A nucleotide mixture that is provided during any one flow may comprise any number of canonical base types (e.g., A, T, G, C, U), such as a single canonical base type, two canonical base types, three canonical base types, four canonical base types or five canonical base types (including T and U). Different types of nucleotide bases may be flowed in any order and/or in any mixture of base types that is useful for sequencing. Various flow-based sequencing systems and methods are described in U.S. Pat. Pub. No. 2022/0170089A1, which is entirely incorporated by reference herein for all purposes. In some cases, nucleotides of different canonical base types may be labeled and detectable at a single frequency (e.g., using the same or different dyes). In other cases, nucleotides of different canonical base types may be labeled and detectable at different frequencies (e.g., using the same or different dyes).

Subsequent to sequencing, the sequencing signals collected and/or generated may be subjected to data analysis. The sequencing signals may be processed to generate base calls and/or sequencing reads. In some cases, the sequencing reads may be processed to generate diagnostics data to the biological sample, or the subject from which the biological sample was derived from. The data analysis may comprise image processing, alignment to a genome or reference genome, training and/or trained algorithms, error correction, and the like.

### EXAMPLES

These examples are provided for illustrative purposes only and are not intended to limit the scope of the claims provided herein.

### Example 1: General Synthetic Principles

Certain examples of the following examples illustrate various methods of making linkers and labeled substrates described herein. It is understood that one skilled in the art may be able to make these compounds by similar methods or by combining other methods known to one skilled in the art. It is also understood that one skilled in the art would be able to make other compounds in a similar manner as described below by using the appropriate starting materials and modifying synthetic routes as needed. In general, starting materials and reagents can be obtained from commercial vendors or synthesized according to sources known to those skilled in the art or prepared as described herein.

Unless otherwise noted, reagents and solvents used in synthetic methods described herein are obtained from commercial suppliers. Anhydrous solvents and oven-dried glassware may be used for synthetic transformations sensitive to moisture and/or oxygen. Yields may not be optimized. Reaction times may be approximate and may not be optimized. Materials and instrumentation used in synthetic procedures may be substituted with appropriate alternatives. Column chromatography and thin layer chromatography (TLC) may be performed on reverse-phase silica gel unless otherwise noted. Nuclear magnetic resonance (NMR) and mass spectra may be obtained to characterize reaction products and/or monitor reaction progress.

This application describes various components that may be covalently linked together, including substrates, linkers, optical moieties, dendrimers, and/or sub-components thereof. It will be appreciated that any component or sub-component may be attached to another component or sub-component according to sources and methods known to those skilled in the art or prepared as described herein to synthesize an intermediate or final molecule, such as a linker, dendrimer, labeling reagent, labeled reagent, or labeled substrate.

In some cases, respective functional groups on two components may be subjected to a reaction to form a covalent bond between the two components. In one example, respective functional groups are subjected to a click reaction. The click reaction may involve using a pair of moieties, a first moiety attached to a first component and a second moiety attached to a second component. The pairs may be any suitable pairs for reactions. Non-limiting examples include Copper (I) catalyzed click: Azide/alkyne reagents; copper-free click:

dibenzocyclooctyne (DBCO)/azide; and copper-free click: trans-cyclooctene (TCO)/tetrazine. The click reaction may be a copper click reaction that comprises the use of copper. Alternatively, the click reaction may be a different click reaction which does not comprise the use of copper. Such reactions may comprise the use of reagents with strained cyclooctenes such as TCO which may react with tetrazines, or cyclooctyne moieties, e.g., DBCO, which may react with azides, e.g., bicyclo[6.1.0]nonyne (BCN), which may react with azides.

### Example 2: Synthesizing Hypₙ

A large order hydroxyproline moiety, Hypₙ (e.g., n>=20, 30, 40, 50, etc.), as used and described herein, may be synthesized by adding two or more smaller order hydroxyproline moieties, Hypₙ (e.g., n>=20, 15, 10, 9, 8, 7, 6, 5, 4, 3, etc.). For example, a Hyp30 is created by adding a Hyp10 and Hyp20. In another example, a Hyp40 is created by adding two Hyp20's. In another example, a Hyp12 is created by adding two Hyp6's. As seen from these examples, the two or more smaller order Hypₙ moieties may or may not be the same lengths.

### Example 3: SUMO1 Labeled Proteins

Provided are example structures of labeled proteins that use SUMO1 proteins. The labeled proteins may be used to label substrates, such as nucleotides, to generate the labeled substrates described herein.

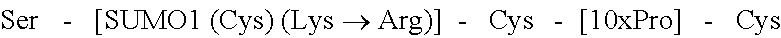

In the above labeled protein, the serine residue at or near the N-terminal is used as the substrate attachment site, all lysine residues in the SUMO1 are modified to arginine residues to prevent potential optical moiety attachment to the lysine, and three cysteine residues, one native to SUMO1 and two appended near the C-terminal are used as optical moiety attachment sites. The two cysteine residues at the C-terminal are separated by a 10-proline polyproline.

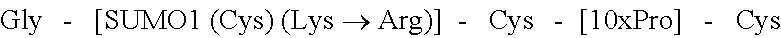

In the above labeled protein, the glycine residue at or near the N-terminal is used as the substrate attachment site, all lysine residues in the SUMO1 are modified to arginine residues to prevent potential optical moiety attachment to the lysine, and three cysteine residues, one native to SUMO1 and two appended near the C-terminal are used as optical moiety attachment sites. The two cysteine residues at the C-terminal are separated by a 10-proline polyproline.

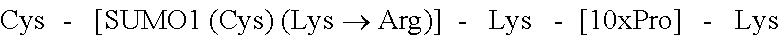

In the above labeled protein, the cysteine residue at or near the N-terminal is used as the substrate attachment site, all lysine residues in the SUMO1 are modified to arginine residues to prevent potential optical moiety attachment to the lysine, and three residues, one cysteine native to SUMO1 and two lysine residues appended near the C-terminal are used as optical moiety attachment sites. The two lysine residues at the C-terminal are separated by a 10-proline polyproline.

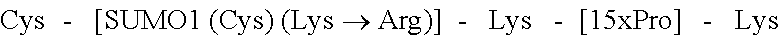

In the above labeled protein, the cysteine residue at or near the N-terminal is used as the substrate attachment site, all lysine residues in the SUMO1 are modified to arginine residues to prevent potential optical moiety attachment to the lysine, and three residues, one cysteine native to SUMO1 and two lysine residues appended near the C-terminal are used as optical moiety attachment sites. The two lysine residues at the C-terminal are separated by a 15-proline polyproline.

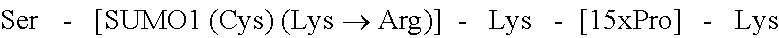

In the above labeled protein, the serine residue at or near the N-terminal is used as the substrate attachment site, all lysine residues in the SUMO1 are modified to arginine residues to prevent potential optical moiety attachment to the lysine, and three residues, one cysteine native to SUMO1 and two lysine residues appended near the C-terminal are used as optical moiety attachment sites. The two lysine residues at the C-terminal are separated by a 15-proline polyproline.

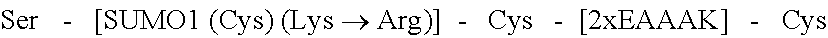

In the above labeled protein, the serine residue at or near the N-terminal is used as the substrate attachment site, all lysine residues in the SUMO1 are modified to arginine residues to prevent potential optical moiety attachment to the lysine, and three cysteine residues, one native to SUMO1 and two appended near the C-terminal are used as optical moiety attachment sites. The two cysteine residues at the C-terminal are separated by 2 EAAAK linkers.

### Example 4: Incorporation of SUMO Labeled Substrates

The following incorporation experiment demonstrates that a nucleotide substrate labeled with a SUMO protein is active as a polymerase substrate.

The test substrate was prepared by conjugating a dUTP to a Atto532 (green dye)-labeled SUMO protein. This conjugate is referred to as dUTP* in this Example.

Two synthetic oligonucleotides were used as templates in this experiment. Each of these oligonucleotides has a hairpin and is labeled with the Pacific Blue (PacBlue) fluorophore at the 5′ end. The two templates comprise the sequence: . . . AₙG . . . PacBlue, where n=2 and n=5, respectively.

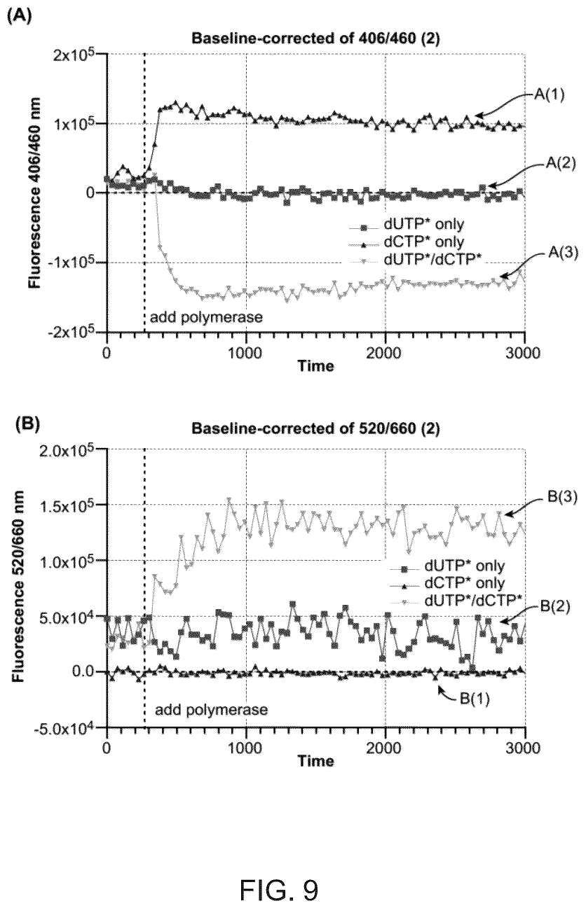

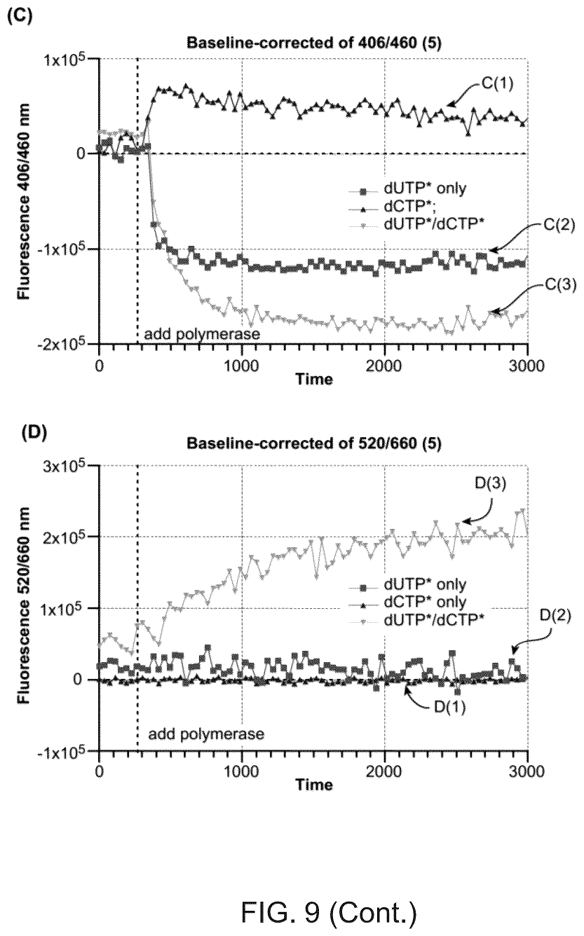

Each template oligonucleotide was prepared in a solution at 10 nM in polymerase assay buffer and aliquoted into three tubes. The following three reagents were added to each set of the three tubes: (1) dCTP-Atto633, to 100 nM; (2) dUTP*, to 600 nM; (3) a mixture of dCTP-Atto633 and dUTP*, to 100 nM and 600 nM, respectively. Then, 50 μL aliquots of the tube contents were placed in individual wells of a 96 well plate. The well plate was placed in a fluorescent plate reader set at 45° C. Fluorescence was monitored using two excitation/emission wavelength settings, 406/460 nm and 520/660 nm, respectively. The reading was interrupted after about 5 minutes and 2 μL of a polymerase dilution were added to a subset of wells, and fluorescence monitoring was re-initiated. The raw data from the plate reader was exported to Graphpad Prism software. Data from the wells not containing the polymerase was subtracted from the wells to which the polymerase had been added to correct for non-enzymatic drifts of the fluorescent signals. The results are plotted in FIG. 9.

Panels (A) and (B) show results for templates with n=2, with wavelength settings of 406/460 nm and 520/660 nm, respectively. Panels (C) and (D) show results for templates with n=5, with wavelength settings of 406/460 nm and 520/660 nm, respectively. The 406/460 nm plot shows changes in the PacBlue channel. The 520/660 nm plot shows the resonance energy transfer channel between the Atto532 of the dUTP* and the Atto633 of the dCTP-Atto633. The vertical dotted line indicates the time of the polymerase addition (about 5 minutes in). Each plot label containing (1), (2), or (3) corresponds to which of the three reagents was added to the template oligonucleotides, as outlined above: (1) dCTP-Atto633; (2) dUTP*; (3) a mixture of dCTP-Atto633 and dUTP*.

From the dCTP-Atto633 “(1)” plots, it can be observed that adding the polymerase results in an initial increase of signals followed by a very slow decrease of signals in the 406/460 nm channel for both templates (see A(1) and C(1)). The initial increase is due to the effect of the polymerase binding to the PacBlue labeled templates, while the following slow decrease is due to a slow misincorporation reaction. The polymerase addition has no effect to the signals in the 520/660 channel in the same wells (see B(1) and D (1)).

From the dUTP* “(2)” plots, it can be observed that adding the polymerase results in a decrease of signals in the 406/460 nm channel for both templates (see A(2) and C(2)), where the decrease is significantly greater for the n=5 template (longer poly A sequence, see C(2)) than for the n=2 template (shorter poly A sequence, see A(2)). The decrease is due to the quenching of the PacBlue. No change in the 520/660 channel is detectable in these wells (see B(2) and D(2))

From the mixture of dCTP-Atto633 and dUTP* “(3)” plots, it can be observed that significant changes in signals occur in both channels. In the 406/460 nm channel, the signals decrease significantly (see A(3) and C(3)). In the 520/660 nm channel, the signals increase significantly (see B(3) and D(3)) due to the sequential incorporation of two or five dUTP* nucleotides followed by the next correct nucleotide, the dCTP-Atto633 nucleotide. The increase is faster in the n=2 template (shorter poly A sequence, see B(3)) than the n=5 template (longer poly A sequence, see D(3)).

The incorporation assay demonstrates that SUMO-labeled dUTP is active as a polymerase substrate.

### Example 5: Incorporation of Streptavidin Labeled Substrates

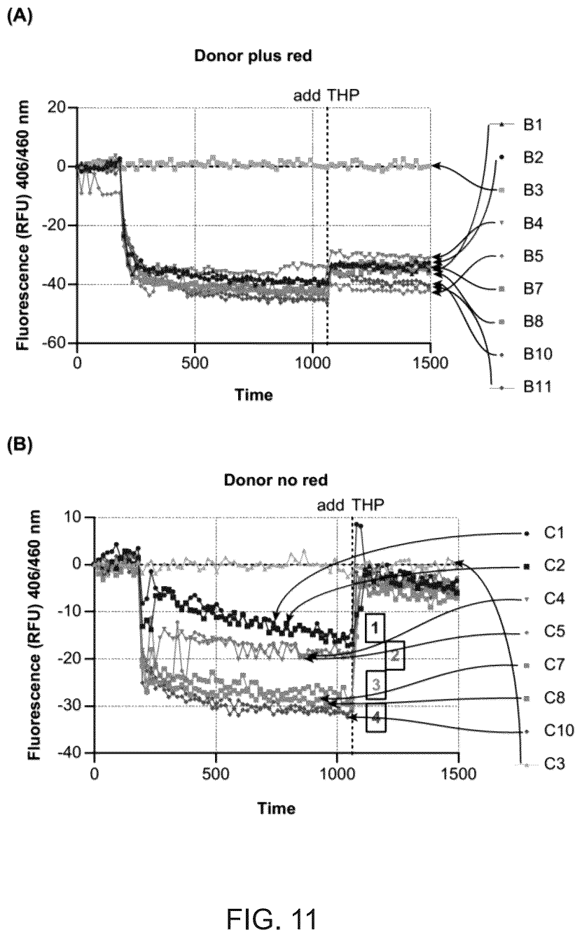

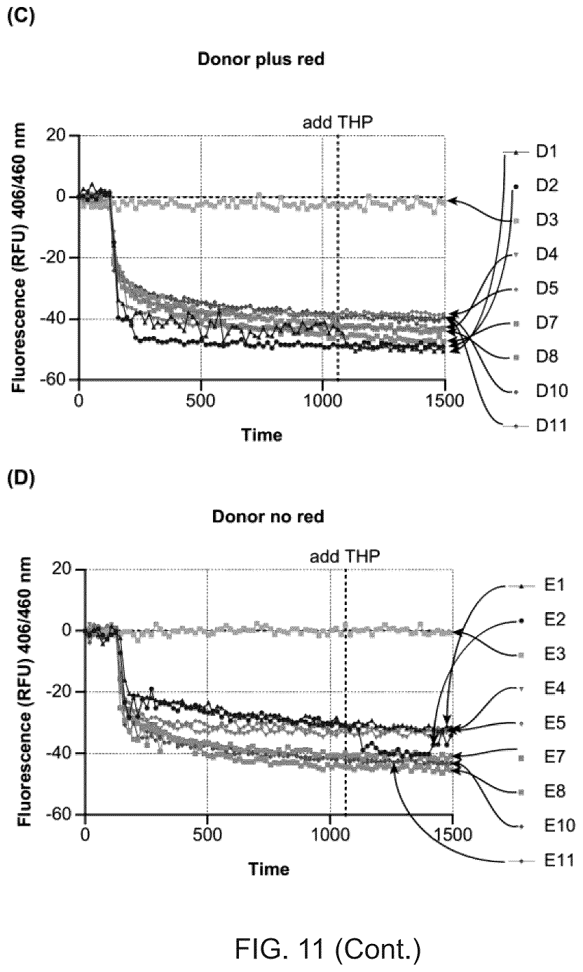

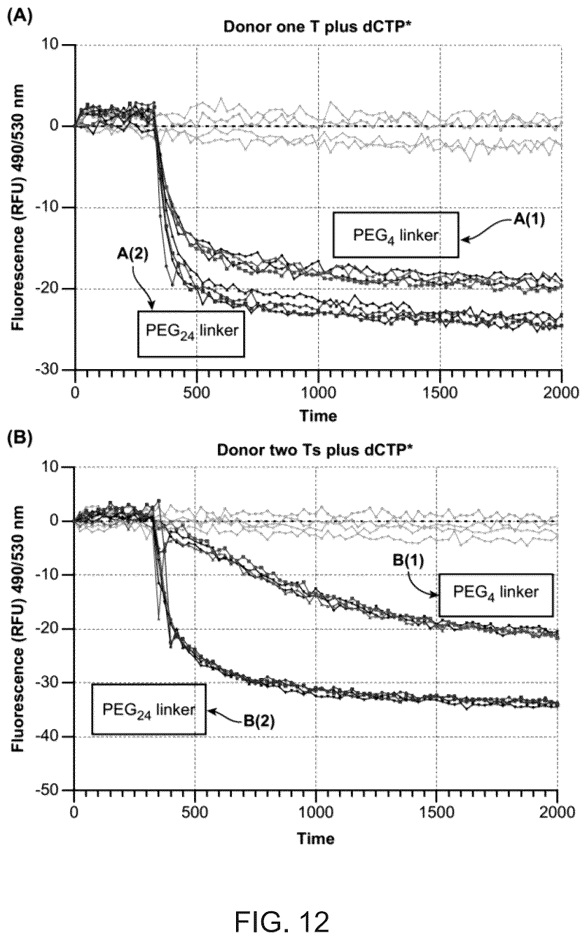

The following incorporation experiments, results shown in FIGS. 11-12, demonstrate that a nucleotide substrate labeled with a streptavidin protein is active as a polymerase substrate.

A first test substrate was prepared by conjugating a biotinylated dATP with a Y cleavable linker (see FIG. 1 for linker) and PEG₂₄ linker to a Atto532-labeled streptavidin. The labeled conjugate has the following structure: dATP-Y-PEG₂₄-biotin-Streptavidin-Atto532 (referred to as dATP* in this Example). A set of four synthetic oligonucleotides was used as templates in this incorporation experiment. The templates comprise the sequence structure of [T]-[GC]-[TTT]-PB, where PB=Pacific Blue, where n=1, 2, 3, or 4. In a first series of assays, both (1) dATP* and (2) non-cleavably-Atto633-labeled dCTP (referred to as dCTP* in this Example) were provided to each template (n=1, 2, 3, or 4) to test for extension activity of primers hybridized thereto. In a second series of assays, dATP* was provided to each template without dCTP*. For both series of assays, after some time, THP, a cleaving agent, was added. The results of the first series of assays and second series of assays are shown in FIG. 11, panels (A) and (B), respectively, each panel plotting Fluorescence (RFU) 406/460 nm vs Time(s), with the vertical dotted line representing the timepoint in which THP was added. For both series of assays, the experiment is designed to detect incorporation of the dATP* via quenching of the PB fluorescence in the template by either the dCTP* (which can only be incorporated subsequent to incorporation of the dATP* opposite the Tn segment in the template) and/or the dATP*. Upon addition of the THP, the Atto532-labeled streptavidin of the dATP* is expected to be cleaved off. As seen in panel (A), where dCTP* was present, the addition of dATP* caused quenching of the PB in the template in a template length-independent manner (most of the quenching attributed to that by the dCTP*) and subsequent addition of THP caused a minor reversal of quenching because the red dye of the dCTP* remained incorporated and the PB fluorescence remained mostly quenched. In panel (A), B3 is control, B1 and B2 are for n=1, B4 and B5 are for n=2, B7 and B8 are forn=3, and B10 and B11 are forn=4. As seen in panel (B), where dCTP* was absent, the addition of dATP* caused quenching of the PB in the template in a template length-dependent manner and subsequent addition of THP caused a mostly complete reversal of quenching as all incorporated dyes were removed and the PB fluorescence was mostly restored. Apparent misincorporation was observed with the n=1 template of A incorporating opposite G, but not for the other templates. In panel (B), C3 is control, C11 and C2 are for n=1, C4 and C5 are for n=2, C7 and C8 are for n=3, and C10 is for n=4.

A second test substrate was prepared by conjugating a biotinylated dATP with a non-cleavable linker (different from the cleavably labeled first test substrate) and PEG₂₄ linker to a Atto532-labeled streptavidin. Specifically, the non-cleavable linker is not cleavable by THP. The labeled conjugate has the following structure: dATP-PEG₂₄-biotin-Streptavidin-Atto532 (referred to as dATP** in this Example). A set of four synthetic oligonucleotides was used as templates in this incorporation experiment. The templates comprise the sequence structure of [T]-[GC]-[TTT]-PB (where PB=Pacific Blue), where n=1, 2, 3, or 4. In a third series of assays, both (1) dATP** and (2) dCTP* were provided to each template (n=1, 2, 3, or 4) to test for extension activity of primers hybridized thereto. In a fourth series of assays, dATP** was provided to each template without dCTP* For both series of third and fourth assays, after some time, THP, a cleaving agent, was added. The results of the third series of assays and fourth series of assays are shown in FIG. 11, panels (C) and (D), respectively, each panel plotting Fluorescence (RFU) 406/460 nm vs Time(s), with the vertical dotted line representing the timepoint in which THP was added. For both series of assays, the experiment is designed to detect incorporation of the dATP* via quenching of the PB fluorescence in the template by either the dCTP* (which can only be incorporated subsequent to incorporation of the dATP* opposite the Tn segment in the template) and/or the dATP*. Upon addition of the THP, the Atto532-labeled streptavidin of the dATP* is not expected to be cleaved off as a non-cleavable linker is used. As seen in panel (C), where dCTP* was present, the addition of dATP** caused quenching of the PB in the template in a template length-independent manner (most of the quenching attributed to that by the dCTP*) and subsequent addition of THP had no effect as the linkers used in these assays are non-cleavable. In panel (C), D3 is control, D1 and D2 are for n=1, D4 and D5 are for n=2, D7 and D8 are for n=3, and D10 and D11 are for n=4. As seen in panel (D), where dCTP* was absent, the addition of dATP** caused quenching of the PB in the template in a template length-dependent manner and subsequent addition of THP similarly had no effect as the linkers used in these assays are non-cleavable. In panel (D), E3 is control, E1 and E2 are for n=1, E4 and E5 are forn=2, E7 and E8 are for n=3, and E10 and E11 are for n=4. Apparent misincorporation was observed with the n=1 template of A incorporating opposite G, but not for the other templates.

A third test substrate was prepared by conjugating a biotinylated dATP with a PEG₄ linker to a Atto633-labeled streptavidin. The labeled conjugate has the following structure: dATP-PEG₄-biotin-Streptavidin-Atto633 (referred to as dATP^{#} in this Example). A fourth test substrate was prepared by conjugating a biotinylated dATP with a PEG₂₄ linker to a Atto633-labeled streptavidin. The labeled conjugate has the following structure: dATP-PEG₂₄-biotin-Streptavidin-Atto633 (referred to as dATP^{##} in this Example). The third test substrate and fourth test substrate have different PEG linker lengths. Two synthetic oligonucleotides were used as templates in this incorporation experiment. The first template and second template comprise the sequence structure of, from 3′ to 5′, [T]-[GC]-[TTT]-_{FL} and [TT]-[GC]-[TTT]-_{FL}, respectively. Incorporation was measured for dATP^{#} and dATP^{##} for each of the first template and the second template. The results are shown in FIG. 12, panel (A) for the first template and panel (B) for the second template, each panel plotting Fluorescence (RFU) 490/530 nm vs Time(s), where the results of the PEG₄ linker on the first template are indicated by A(1) and on the second template are indicated by B(1), and where the results of the PEG₂₄ linker on the first template are indicated by A(2) and on the second template are indicated by B(2). As can be seen in panels (C) and (D), significantly faster incorporation rates were observed for the fourth substrate with the longer PEG₂₄ linker (see A(2) and B(2)) compared to the third substrate with the PEG₄ linker (see A(1) and B(1)).

### Example 6: Using Streptavidin Labeled Substrates with FRET

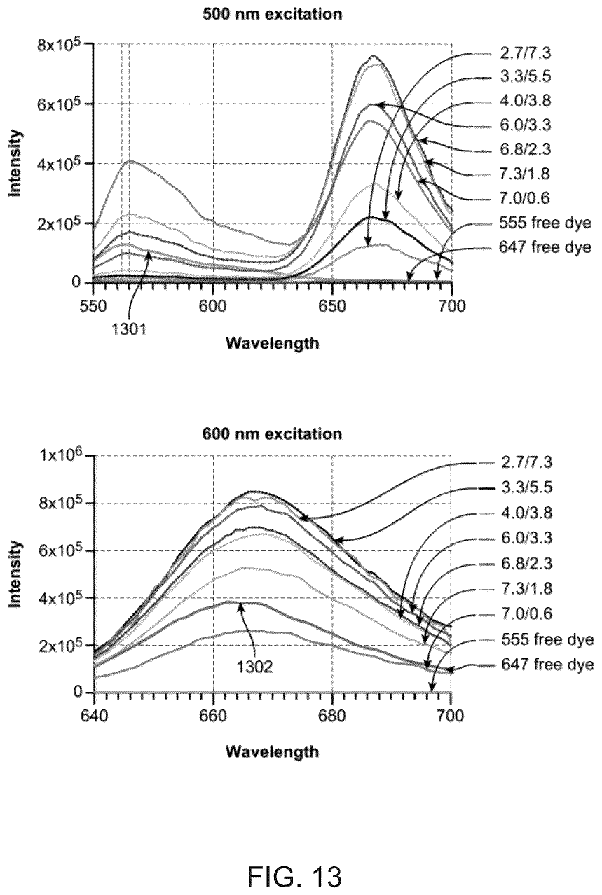

FIG. 13 illustrates the intensity vs wavelength plot for various labelled streptavidin scaffolds, where streptavidin is labelled with different ratios of red dyes (AZ647) and green dyes (AZ555), for excitation at 500 nm (top panel) and 600 nm (bottom panel). Streptavidin is labelled with the following red dyes/green dyes per streptavidin: 2.7/7.3; 3.3/5.5; 4.0/3.8; 6.0/3.3; 6.8/2.3; 7.3/1.8; 7.0/0.6. Also illustrated on the chart is the free AZ555 dye (1301) and free AZ647 dye (1302).

Based on these plots, a FRET-based detection method may comprise a single excitation channel in the green wavelength range and three emission channels (e.g., in green, red, and ‘redder’ wavelength range). In the context of sequencing, the four canonical base types of nucleotides may be encoded in one example by any four of the five labelling schemes: (1) green dye only, (2) green dye and red dye, (3) green dye and redder dye, and (4) green dye, red dye, and redder dye, and (5) no dye. Different methods of labelling a single substrate with multiple optical moieties (the same type or different types) are described elsewhere herein.

### Example 7: Multi-Dye Fluorescence

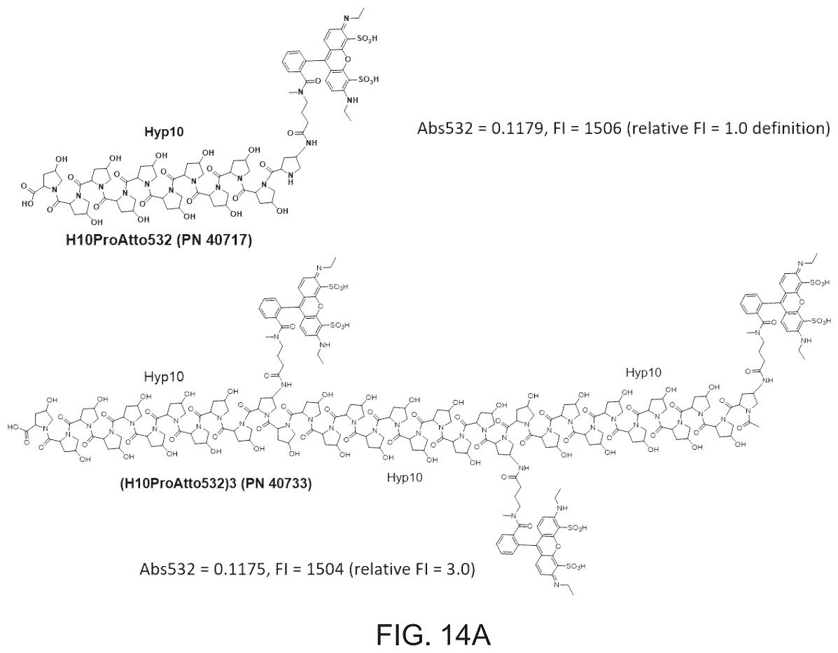

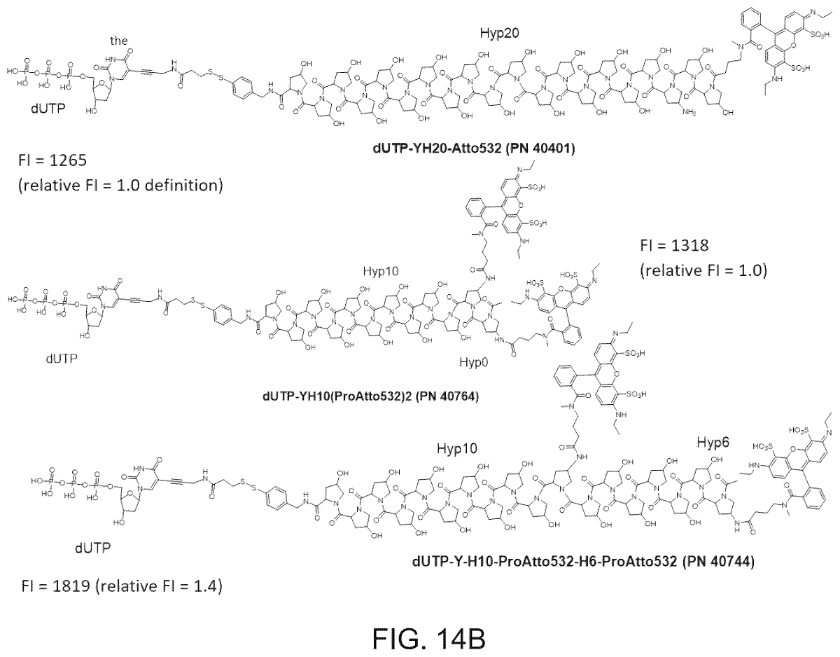

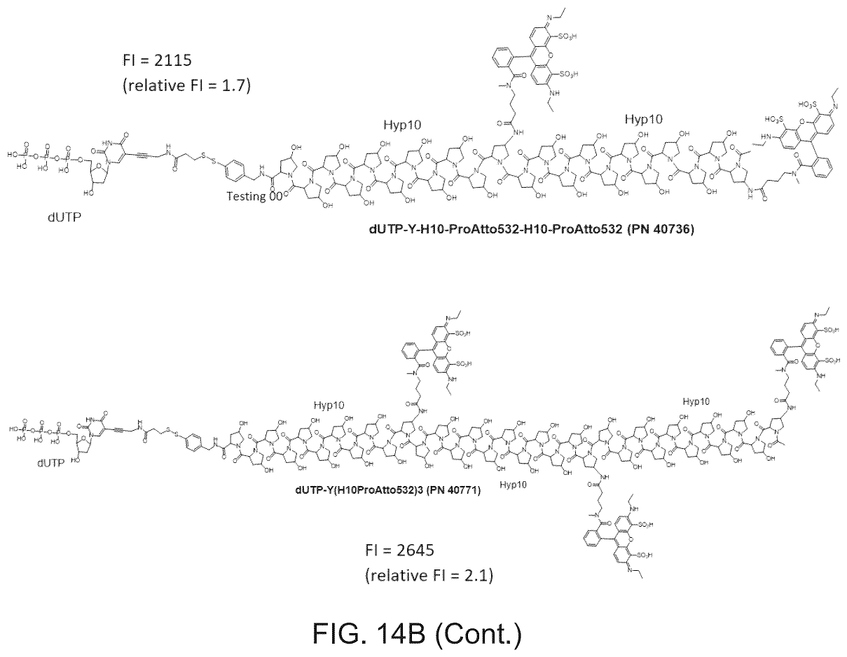

FIGS. 14A-B illustrate the relative fluorescence intensity (FI) measured for linkers and substrates labeled with different number of dyes. This assay demonstrates that fluorescence intensity measured from labeled substrates generally increases with increasing number of dye moieties labelling said labeled substrates, and the effect of quenching between the multiple dyes and/or the substrate (e.g., base) decreases with larger spacing between the different entities.

A first assay compared the relative FI values of labeled linkers that are not attached to substrates (e.g., bases). FIG. 14A shows in the top, a first labeled linker with structure: H10ProAtto532, where a Hyp10 linker is labeled at one end with a single Atto532 dye moiety, and in the bottom, a second labeled linker with structure: (H10ProAtto532)₃, where three units, each with a Hyp₁₀ linker and one Atto532 dye moiety, are joined together to form a linker with three Atto532 dye moieties. The first labeled linker with the single dye moiety yielded an absolute FI value of 1506 units, which is defined in relative FI as 1.0 unit. The relative FI adjusts for concentration of the molecules in the measured solution. The second labeled linker with the three dye moieties yielded an absolute FI value of 1504 units, which converts to a relative FI of 3.0 units. The results confirmed a near linear correlation between number of dyes and relative FI.

A second assay compared the relative FI values of dUTP substrates that are labeled with dye(s) via a Y linker and hypn linker (see FIG. 1), the dye(s) being spaced apart differently on the linker. FIG. 14B shows labeled substrates each at 852 nM concentrations from top to bottom, a first labeled substrate with structure: dUTP-YH20-Atto532, where the dUTP substrate and single Atto532 dye are spaced by a Hyp20 moiety; a second labeled substrate with structure: dUTP-YH10(ProAtto532)₂, where the dUTP substrate is spaced from two Atto532 dyes by a Hyp10 moiety, the two Atto532 dyes adjacent to each other (Hyp0 spacing); a third labeled substrate with structure: dUTP-Y-H10-ProAtto532-H6-ProAtto532, where the dUTP substrate is spaced from the closest first Atto532 dye by a Hyp10 moiety and the first and second Atto532 dyes are spaced by a Hyp6 moiety; a fourth labeled substrate with structure: dUTP-Y-H10-ProAtto532-H10-ProAtto532, where the dUTP substrate is spaced from the closest first Atto532 dye by a Hyp10 moiety and the first and second Atto532 dyes are spaced by a Hyp10 moiety; and a fifth labeled substrate with structure: dUTP-Y(H10ProAtto532)₃, where the dUTP substrate is spaced from the closest first Atto532 dye by a Hyp10 moiety, the first and second Atto532 dyes are spaced by a Hyp10 moiety, and the second and third Atto532 dyes are spaced by a Hyp10 moiety. The first labeled substrate with a single dye moiety yielded an absolute FI value of 1265 units, which is defined in relative FI as 1.0 unit. Since the solutions in FIG. 14B each have a concentration of 852 nM, the relative FI compares the fluorescent intensity of each labeled substrate with the top labeled substrate. The second labeled substrate with two dye moieties yielded an absolute FI value of 1318 units, which converts to a relative FI of 1.0 unit. That is, quenching caused by two dyes that are disposed directly adjacent to each other (e.g., no spacing) was substantial enough to negate the presence of an additional dye compared to the first labeled substrate. The third labeled substrate with two dye moieties yielded an absolute FI value of 1819 units, which converts to a relative FI of 1.4 units. That is, quenching caused by two dyes that are disposed farther from each other (e.g., Hyp0 vs Hyp6) was reduced and thus resulted in increased relative FI compared to the second labeled substrate. The fourth labeled substrate with two dye moieties yielded an absolute FI value of 2115 units, which converts to a relative FI of 1.7 units. That is, quenching caused by two dyes that are disposed farther from each other (e.g., Hyp6 vs Hyp10) was additionally reduced and thus resulted in increased relative FI compared to the third labeled substrate. The fifth labeled substrate with three dyes that are each disposed substantially from each other (e.g., Hyp10) yielded an absolute FYI value of 2645, which converts to a relative FI of 2.1 units. That is, an increased number of dyes, when substantially spaced from other dyes, generally resulted in increased relative FI.

Additionally, the disparity in linear correlation between the FI and number of dyes from the first assay (linearity observed) to the second assay (linearity not observed) appears to be due to the presence of the substrate at the end of the linker, which contributes to quenching of one or more dyes.

### Example 8: Multi-Dye Dendrimer Fluorescence

Different multiply-labeled dendrimers were assayed to determine fluorescence behavior. The molecule illustrated in FIG. 6C, of a dendrimer labeled with 8 Atto532 dyes each closely spaced with each other (short dendrimeric branch; minimal distance between the dendrimer core and each of the dyes), was found to exhibit almost no fluorescence. In contrast, the bottom left molecule illustrated in FIG. 6B, of a dendrimer labeled with 4 Kam dyes each spaced from the core by at least a rigid hyp10 segment (relatively longer dendrimeric branch), was found to exhibit about 3.2 times more relative fluorescent intensity than the top right molecule illustrated in FIG. 6B, of a dendron comprising a single Kam dye attached at the end of the hyp10 linker. These assays generally demonstrate that fluorescence intensity measured from labeled dendrimers generally increases with increasing number of dye moieties on said labeled dendrimers on condition the dye moieties are sufficiently spaced to prevent detrimental quenching between the multiple dye moieties.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### NUMBERED EMBODIMENTS

### Embodiment 1

A sequencing reagent, comprising:
a nucleotide;
a linker;
a fluorescent dye moiety; and
a dendrimer comprising a plurality of dendrimer branches each comprising a repeating number of polyethylene glycol (PEG) or hydroxyproline, wherein said linker is attached to a nucleobase of said nucleotide and said dendrimer, and wherein (i) two dendrimer branches of said plurality of dendrimer branches are attached to a dendrimer core of said dendrimer and said fluorescent dye moiety is disposed between said two dendrimer branches and attached to said dendrimer core or (ii) two nth order dendrimer branches of said plurality of dendrimer branches are attached to a (n−1)th dendrimer branch of said dendrimer and said fluorescent dye moiety is disposed between said two nth order dendrimer branches and attached to said (n−1)th dendrimer branch.

### Embodiment 2

The sequencing reagent of embodiment 1, wherein said two dendrimer branches or two nth order dendrimer branches are terminated by a water soluble group.

### Embodiment 3

The sequencing reagent of embodiment 2, wherein said water soluble group comprises sulfonic acid.

### Embodiment 4

The sequencing reagent of any of claims 1-3, wherein said sequencing reagent comprises at least two fluorescent dye moieties, wherein each respective fluorescent dye of said at least two fluorescent dye moieties (i) is disposed between a respective pair of dendrimer branches, wherein each respective pair of dendrimer branches and each respective fluorescent dye are attached to said dendrimer core or (ii) is disposed between a respective pair of nth order dendrimer branches, wherein each respective pair of nth order dendrimer branches and each respective fluorescent dye are attached to a respective (n−1)th dendrimer branch of said dendrimer.

### Embodiment 5

The sequencing reagent of embodiment 4, wherein said sequencing reagent comprises at least four fluorescent dye moieties.

### Embodiment 6

The sequencing reagent of embodiment 4, wherein said sequencing reagent comprises at least eight fluorescent dye moieties.

### Embodiment 7

A sequencing reagent, comprising:
a nucleotide;
a linker;
a fluorescent dye moiety; and
a dendrimer comprising a plurality of dendrimer branches each comprising a repeating number of a polymer, polyethylene glycol (PEG), or hydroxyproline, wherein said linker is attached to a nucleobase of said nucleotide and said dendrimer, and wherein said fluorescent dye moiety is attached to a distal end of a highest order dendrimer branch of said plurality of dendrimer branches relative to a dendrimer core of said dendrimer.

### Embodiment 8

The sequencing reagent of embodiment 7, wherein said sequencing reagent comprises at least two fluorescent dye moieties, wherein each respective fluorescent dye of said at least two fluorescent dye moieties is attached to a respective distal end of a respective highest order dendrimer branch of said plurality of dendrimer branches relative to said dendrimer core.

### Embodiment 9

The sequencing reagent of embodiment 8, wherein said sequencing reagent comprises at least four fluorescent dye moieties.

### Embodiment 10

The sequencing reagent of embodiment 9, wherein said sequencing reagent comprises at least eight fluorescent dye moieties.

### Embodiment 11

A method, comprising: using said sequencing reagent of any one of claims 1-10 in a sequencing reaction comprising providing said sequencing reagent to a template nucleic acid molecule hybridized to an extending sequencing primer molecule.

### Embodiment 12

The method of embodiment 11, further comprising incorporating said nucleotide into said extending sequencing primer molecule and detecting said fluorescent dye moiety.

### Embodiment 13

A composition, comprising:
a sequencing reagent, comprising:
   a template nucleic acid molecule;
   a linker; and
   a dendrimer comprising a plurality of dendrimer branches each comprising a repeating number of a polymer, polyethylene glycol (PEG), or hydroxyproline, wherein said linker is attached to said template nucleic acid molecule and said dendrimer.

### Embodiment 14

The composition of embodiment 13, wherein said dendrimer has at least four orders of dendrimer branches.

### Embodiment 15

The composition of embodiment 14, wherein said dendrimer has at least six orders of dendrimer branches.

### Embodiment 16

The composition of embodiment 15, wherein said dendrimer has at least eight orders of dendrimer branches.

### Embodiment 17

The composition of embodiment 16, wherein said dendrimer has at least ten orders of dendrimer branches.

### Embodiment 18

The composition of any one of claims 13-17, wherein highest order dendrimer branches of said plurality of dendrimer branches are terminated by a water soluble group.

### Embodiment 19

The composition of embodiment 18, wherein said water soluble group comprises sulfonic acid.

### Embodiment 20

The composition of any one of claims 13-19, wherein said sequencing reagent is in solution and not immobilized to a substrate bigger than 1 mm² in surface area.

### Embodiment 21

The composition of any one of claims 13-19, wherein said sequencing reagent is immobilized to a substrate bigger than 1 mm² in surface area.

### Embodiment 22

The composition of embodiment 21, wherein said sequencing reagent is immobilized to a substrate bigger than 1000 mm² in surface area.

### Embodiment 23

A method for spatially separating objects on a substrate, comprising: loading and immobilizing a plurality of dendrimers as a layer on said substrate, wherein a first dendrimer of said plurality of dendrimers is attached to a nucleic acid template molecule, and wherein said first dendrimer comprises a plurality of dendrimeric branches that spaces said nucleic acid template molecule from an additional nucleic acid template molecule attached to a second dendrimer immobilized adjacent to said dendrimer.

### Embodiment 24

The method of embodiment 23, further comprising sequencing said template nucleic acid molecule.

### Embodiment 25

The method of embodiment 24, wherein said sequencing comprises extending a sequencing primer hybridized to said nucleic acid template molecule by providing nucleotide reagents, washing away unincorporated nucleotides from a reaction space, and detecting signals indicative of incorporation, or lack thereof, of said nucleotide reagents.

### Embodiment 26

A method for spatially separating objects on a substrate, comprising: loading and immobilizing a plurality of dendrimers attached to a plurality of binding agents as a layer on said substrate, wherein a first dendrimer of said plurality of dendrimers is attached to a binding agent, and wherein said first dendrimer comprises a plurality of dendrimeric branches that spaces said binding agent from an additional binding agent attached to a second dendrimer immobilized adjacent to said dendrimer; and contacting said plurality of dendrimers immobilized to said substrate with a plurality of template nucleic acid molecules, thereby binding template nucleic acid molecules of said plurality of template nucleic acid molecules to said plurality of binding agents.

### Embodiment 27

The method of embodiment 26, further comprising sequencing said template nucleic acid molecule.

### Embodiment 28

The method of embodiment 27, wherein said sequencing comprises extending a sequencing primer hybridized to said nucleic acid template molecule by providing nucleotide reagents, washing away unincorporated nucleotides from a reaction space, and detecting signals indicative of incorporation, or lack thereof, of said nucleotide reagents.

### Embodiment 29

A labeling reagent, comprising:
a protein;
a predetermined substrate attachment site configured to attach said protein to a substrate; and
a predetermined optical moiety attachment site configured to attach said protein to an optical moiety.

### Embodiment 30

The labeling reagent of embodiment 29, wherein said labeling reagent comprises a single predetermined substrate attachment site.

### Embodiment 31

The labeling reagent of any one of claims 29-30, wherein said labeling reagent comprises at least two predetermined optical moiety attachment sites.

### Embodiment 32

The labeling reagent of embodiment 31, wherein said labeling reagent comprises at least three predetermined optical moiety attachment sites.

### Embodiment 33

The labeling reagent of any one of claims 31-32, wherein two of said at least two predetermined optical moiety attachment sites are separated by a polyproline or at least one EAAAK linker moiety.

### Embodiment 34

The labeling reagent of any one of claims 29-33, wherein said predetermined substrate attachment site is an amino acid residue native to said protein.

### Embodiment 35

The labeling reagent of any one of claims 29-33, wherein said predetermined substrate attachment site is an engineered amino acid residue that is mutated within or coupled to said protein.

### Embodiment 36

The labeling reagent of any one of claims 29-35, wherein said predetermined substrate attachment site is selected from the group consisting of glycine, methionine, arginine, cysteine, tryptophan, tyrosine, lysine, serine and histidine amino acid residues.

### Embodiment 37

The labeling reagent of embodiment 36, wherein said predetermined substrate attachment site is selected from the group consisting of glycine, cysteine, and serine amino acid residues.

### Embodiment 38

The labeling reagent of any one of claims 29-37, wherein said predetermined optical moiety attachment site is an amino acid residue native to said protein.

### Embodiment 39

The labeling reagent of any one of claims 29-37, wherein said predetermined optical moiety attachment site is an engineered amino acid residue that is mutated within or coupled to said protein.

### Embodiment 40

The labeling reagent of any one of claims 29-39, wherein said predetermined optical moiety attachment site is selected from the group consisting of glycine, methionine, arginine, cysteine, tryptophan, tyrosine, lysine, serine and histidine amino acid residues.

### Embodiment 41

The labeling reagent of embodiment 40, wherein said predetermined optical moiety attachment site is selected from the group consisting of cysteine and lysine amino acid residues.

### Embodiment 42

The labeling reagent of any one of claims 29-41, wherein said protein is an engineered protein that has a mutation or deletion of at least one native amino acid residue.

### Embodiment 43

The labeling reagent of any one of claims 29-42, wherein said protein comprises at most 500 amino acid residues.

### Embodiment 44

The labeling reagent of any one of claims 29-43, wherein said protein has a molecular mass of at most 50 kilodaltons (kDa).

### Embodiment 45

The labeling reagent of any one of claims 29-44, wherein a distance between a N-terminus and a C-terminus of said protein is at least about 25 Angstroms (Å).

### Embodiment 46

The labeling reagent of any one of claims 29-45, wherein said predetermined substrate attachment site is disposed within at most 10 amino acid residues of said N-terminus of said protein.

### Embodiment 47

The labeling reagent of any one of claims 29-46, wherein said predetermined optical moiety attachment site is disposed within at most 10 amino acid residues of said C-terminus of said protein.

### Embodiment 48

The labeling reagent of any one of claims 29-47, wherein said protein is a small ubiquitin-like modifier (SUMO) proteins, maltose-binding proteins (MBP), streptavidin, or thioredoxin.

### Embodiment 49

A labeled substrate, comprising:
a substrate;
an optical moiety; and
said labeling reagent of any one of claims 29-48, wherein said substrate is attached to said predetermined substrate attachment site and said optical moiety is attached to said predetermined optical moiety attachment site.

### Embodiment 50

The labeled substrate of embodiment 49, wherein said substrate comprises a nucleotide.

### Embodiment 51

The labeled substrate of any one of claims 49-50, further comprising a cleavable group between said substrate and said predetermined substrate attachment site.

### Embodiment 52

The labeled substrate of embodiment 51, wherein said cleavable group is selected from said group consisting of an azidomethyl group, a disulfide bond, a hydrocarbyldithiomethyl group, and a 2-nitrobenzyloxy group.

### Embodiment 53

The labeled substrate of any one of claims 49-52, wherein said labeling reagent comprise a plurality of optical moiety attachment sites and wherein said labeled substrate further comprise a plurality of optical moieties attached to said plurality of optical moiety attachment sites.

### Embodiment 54

A labeling reagent, comprising:
a fluorescent dye moiety; and
a linker that is connected to said fluorescent dye moiety and configured to couple to a substrate, wherein said linker comprises a polymer linker moiety selected from the group consisting of:

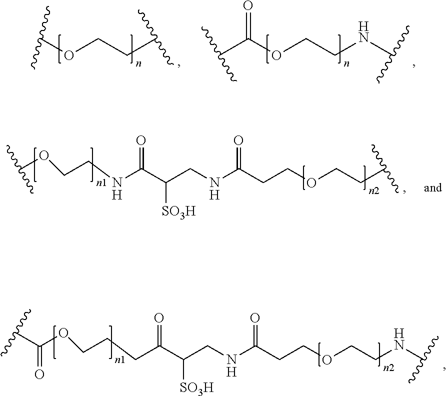

wherein each of n, n1, and n2 is a positive integer.

### Embodiment 55

A labeling reagent, comprising:
a fluorescent dye moiety; and
a linker that is connected to said fluorescent dye moiety and configured to couple to a substrate, wherein said linker comprises:
   (i) a cleavable moiety selected from the group consisting of:

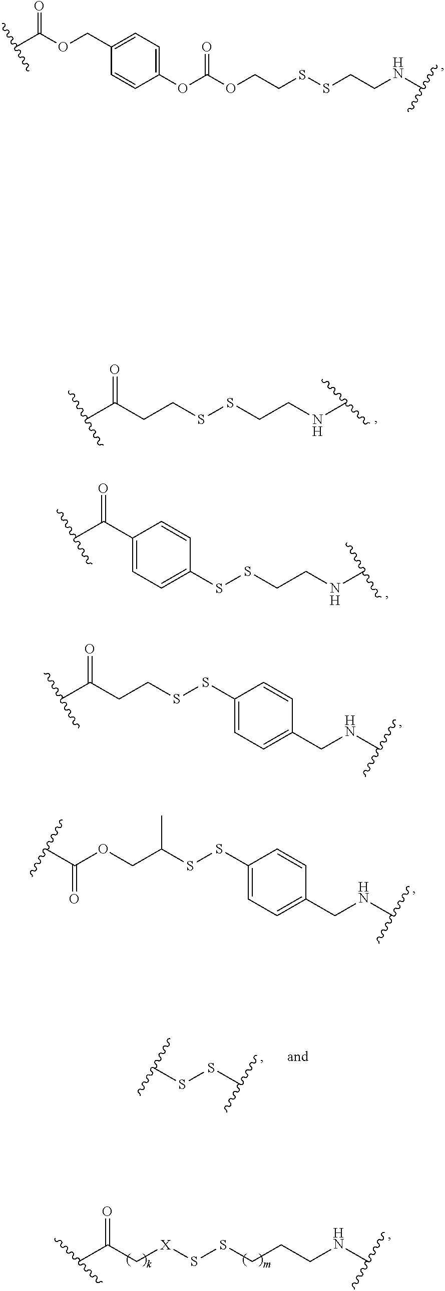

wherein X is a bond or 1,4-phenylene, wherein k is 0, 1, or 2, and wherein m is 0, 1, 2, 3, or 4; and
(ii) a polymer linker moiety selected from the group consisting of:

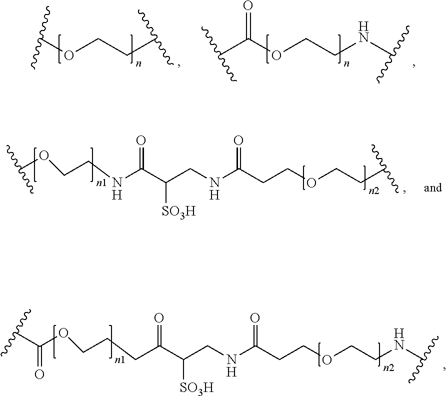

wherein each of n, n1, and n2 is a positive integer.

### Embodiment 56

The labeling reagent of claim 54 or 55, wherein n or (n1+n2) is 8 or greater.

### Embodiment 57

The labeling reagent of embodiment 56, wherein n or (n1+n2) is 75 or greater.

### Embodiment 58

The labeling reagent of embodiment 57, wherein n or (n1+n2) is 100 or greater.

### Embodiment 59

The labeling reagent of any one of claims 54-58, wherein said linker further comprises a moiety selected from the group consisting of:

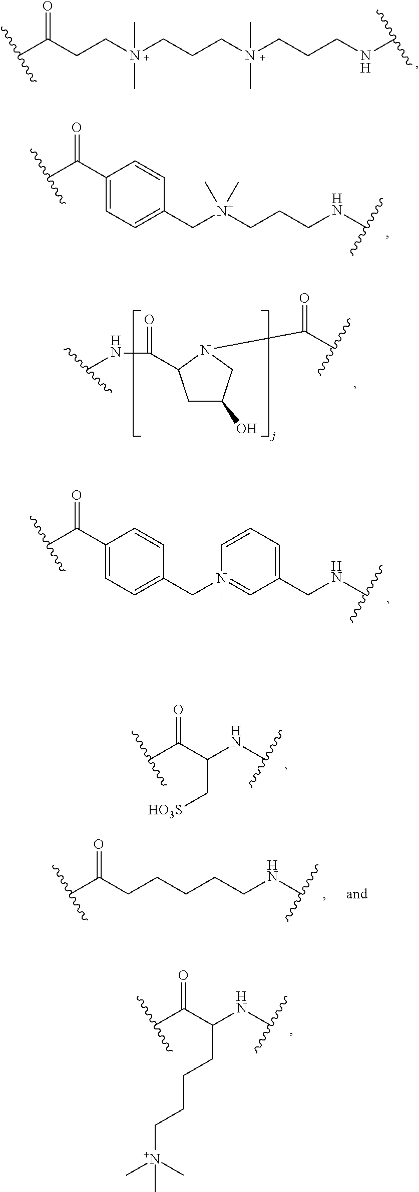

where j is a positive integer.

### Embodiment 60

The labeling reagent of any one of claims 54-59, wherein said linker further comprises one or more glycine moieties.

### Embodiment 61

The labeling reagent of any one of claims 54-60, wherein said linker provides an average physical separation between said fluorescent dye moiety and said substrate of at least 30 Angstroms (Å).

### Embodiment 62

The labeling reagent of any one of claims 54-61, wherein said linker provides an average physical separation between said fluorescent dye moiety and said substrate of at least 60 Angstroms (Å).

### Embodiment 63

The labeling reagent of any one of claims 54-62, wherein said linker further comprises a cleavable group selected from the group consisting of an azidomethyl group, a disulfide bond, a hydrocarbyldithiomethyl group, and a 2-nitrobenzyloxy group.

### Embodiment 64

The labeling reagent of embodiment 63, wherein said cleavable group is cleavable by application of one or more members of the group consisting of tris(2-carboxyethyl) phohsphine (TCEP), dithiothreitol (DTT), tetrahydropyranyl (THP), ultraviolet (UV) light, and a combination thereof.

### Embodiment 65

The labeling reagent of any one of claims 54-64, wherein said substrate is a nucleotide, polynucleotide, protein, lipid, cell, saccharide, polysaccharide, or antibody.

### Embodiment 66

The labeling reagent of embodiment 65, wherein said substrate is a nucleotide and said labeling reagent is configured to attach to a nucleobase of said nucleotide.

### Embodiment 67

The labeling reagent of embodiment 65, wherein said substrate is a protein.

### Embodiment 68

The labeling reagent of any one of claims 54-67, wherein said linker comprises two or more linker branches each coupled to a dendrimer core, wherein each of said two or more linker branches comprises

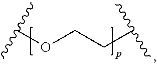

where p is a positive integer, and wherein said dendrimer core is attached to said fluorescent dye moiety.

### Embodiment 69

The labeling reagent of embodiment 68, wherein a linker branch of said two or more linker branches is terminated by a water soluble group.

### Embodiment 70

The labeling reagent of embodiment 69, wherein said water soluble group comprises sulfonic acid.

### Embodiment 71

The labeling reagent of embodiment 70, wherein said water soluble group comprises three sulfonic acid moieties.

### Embodiment 72

The labeling reagent of any one of claims 68-71, wherein each of said two or more linker branches is terminated by a respective water soluble group.

### Embodiment 73

The labeling reagent of any one of claims 68-72, wherein p is at least 8.

### Embodiment 74

A labeled substrate, comprising:
said substrate; and
said labeling reagent of any one of claims 54-73 that is coupled to said substrate.

### Embodiment 75

The labeled substrate of embodiment 74, wherein said substrate is a nucleotide, polynucleotide, protein, lipid, cell, saccharide, polysaccharide, or antibody.

### Embodiment 76

The labeled substrate of embodiment 75, wherein said substrate is a nucleotide and said labeling reagent is configured to attach to a nucleobase of said nucleotide.

### Embodiment 77

The labeled substrate of embodiment 76, wherein said substrate is a protein.

### Embodiment 78

A method, comprising: using said labeling reagent of any one of claims 29-48 and 54-73 in a sequencing reaction comprising providing said labeling reagent to a template nucleic acid molecule hybridized to an extending sequencing primer molecule, wherein said linker of said labeling reagent is coupled to a nucleobase of a nucleotide substrate.

### Embodiment 79

The method of embodiment 78, further comprising incorporating said nucleotide substrate into said extending sequencing primer molecule and detecting said fluorescent dye moiety.

1. A sequencing reagent, comprising:
a nucleotide;
a linker;
a fluorescent dye moiety; and
a dendrimer comprising a plurality of dendrimer branches each comprising a repeating number of polyethylene glycol (PEG) or hydroxyproline, wherein said linker is attached to a nucleobase of said nucleotide and said dendrimer, and wherein (i) two dendrimer branches of said plurality of dendrimer branches are attached to a dendrimer core of said dendrimer and said fluorescent dye moiety is disposed between said two dendrimer branches and attached to said dendrimer core or (ii) two nth order dendrimer branches of said plurality of dendrimer branches are attached to a (n−1)th dendrimer branch of said dendrimer and said fluorescent dye moiety is disposed between said two nth order dendrimer branches and attached to said (n−1)th dendrimer branch.

2. The sequencing reagent of claim 1, wherein said two dendrimer branches or two nth order dendrimer branches are terminated by a water soluble group.

3. The sequencing reagent of claim 2, wherein said water soluble group comprises sulfonic acid.

4. The sequencing reagent of claim 1, wherein said sequencing reagent comprises at least two fluorescent dye moieties, wherein each respective fluorescent dye of said at least two fluorescent dye moieties (i) is disposed between a respective pair of dendrimer branches, wherein each respective pair of dendrimer branches and each respective fluorescent dye are attached to said dendrimer core or (ii) is disposed between a respective pair of nth order dendrimer branches, wherein each respective pair of nth order dendrimer branches and each respective fluorescent dye are attached to a respective (n−1)th dendrimer branch of said dendrimer.

5. The sequencing reagent of claim 4, wherein said sequencing reagent comprises at least four fluorescent dye moieties.

6. The sequencing reagent of claim 4, wherein said sequencing reagent comprises at least eight fluorescent dye moieties.

7. A sequencing reagent, comprising:
a nucleotide;
a linker;
a fluorescent dye moiety; and
a dendrimer comprising a plurality of dendrimer branches each comprising a repeating number of a polymer, polyethylene glycol (PEG), or hydroxyproline, wherein said linker is attached to a nucleobase of said nucleotide and said dendrimer, and wherein said fluorescent dye moiety is attached to a distal end of a highest order dendrimer branch of said plurality of dendrimer branches relative to a dendrimer core of said dendrimer.

8. The sequencing reagent of claim 7, wherein said sequencing reagent comprises at least two fluorescent dye moieties, wherein each respective fluorescent dye of said at least two fluorescent dye moieties is attached to a respective distal end of a respective highest order dendrimer branch of said plurality of dendrimer branches relative to said dendrimer core.

9. The sequencing reagent of claim 8, wherein said sequencing reagent comprises at least four fluorescent dye moieties.

10. The sequencing reagent of claim 9, wherein said sequencing reagent comprises at least eight fluorescent dye moieties.

11.-79. (canceled)

80. The sequencing reagent of claim 7, wherein said linker further comprises a cleavable moiety selected from the group consisting of:

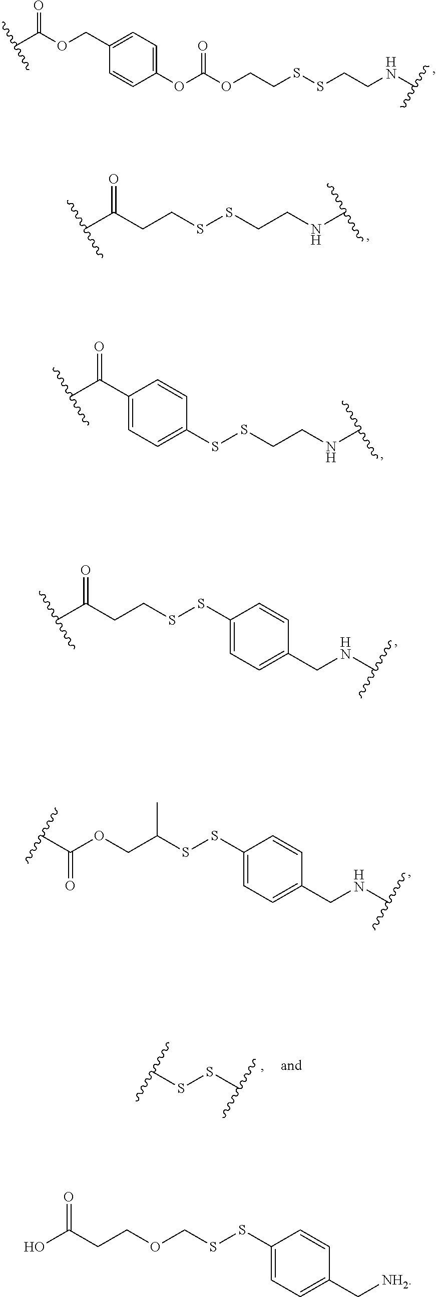

81. The sequencing reagent of claim 7, wherein said plurality of dendrimer branches comprises a polymer selected from the group consisting of 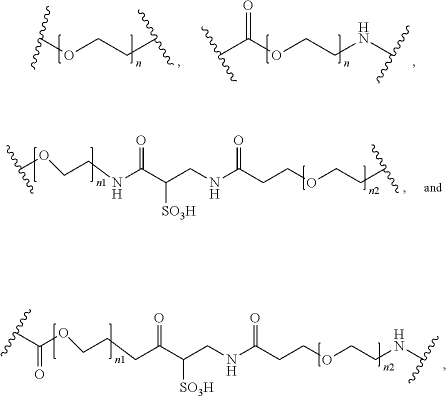
wherein each of n, n1, and n2 is a positive integer.

82. The sequencing reagent of claim 81, wherein n or (n1+n2) is 8 or greater.

83. The sequencing reagent of claim 1, wherein said dendrimer has at least four orders of dendrimer branches.

84. The sequencing reagent of claim 1, wherein a highest order dendrimer branch of said plurality of dendrimer branches is terminated by a branch terminator group.

85. The sequencing reagent of claim 84, wherein said branch terminator group comprises a negative charge.

86. The sequencing reagent of claim 1, wherein said linker further comprises a cleavable moiety selected from the group consisting of:

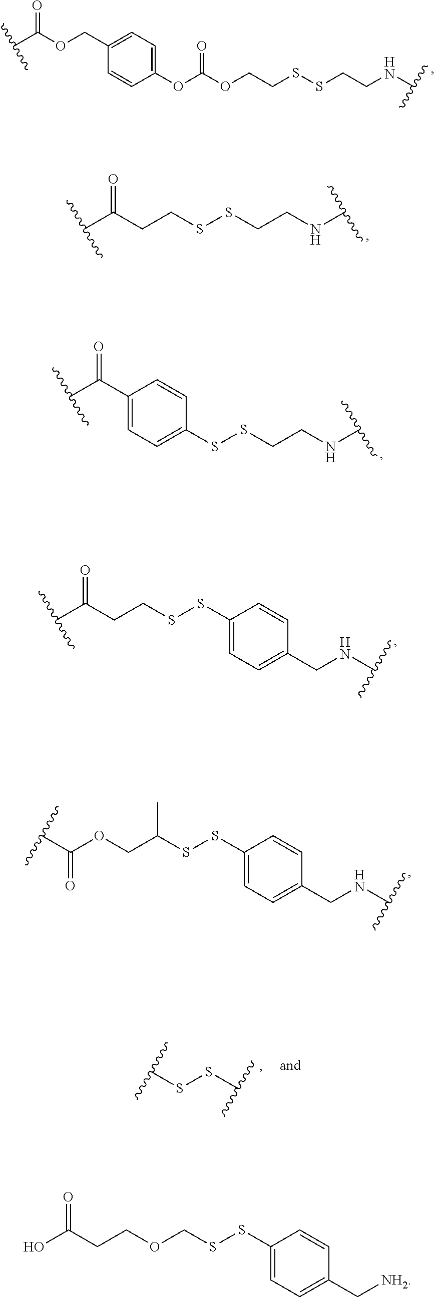

87. The sequencing reagent of claim 1, wherein a dendrimer branch of said plurality of dendrimer branches comprises at least 10 hydroxyproline residues.

88. The sequencing reagent of claim 1, wherein a maximum diameter of the dendrimer is at least 250 nm.

89. The sequencing reagent of claim 88, wherein the maximum diameter of the dendrimer is at least 500 nm.
